(19) United States
(12) Patent Application Publication
(10) Pub. No.: US 2026/0248738 A1
(43) Pub. Date: Aug. 27, 2026

(54) INTRANASAL DELIVERY OF TIRZEPATIDE TO TREAT OBESITY

(71) Applicant: THE TRUSTEES OF COLUMBIA UNIVERSITY IN THE CITY OF NEW YORK

(72) Inventors: Kam W. LEONG, New York, NY (US); Li QIANG, New York, NY (US); Suwan DING, New York, NY (US); Qianfen WAN, New York, NY (US)

(21) Appl. No.: 19/643,702

(22) Filed: Apr. 9, 2026

Related U.S. Application Data
(63) Continuation-in-part of application No. PCT/US24/50578, filed on Oct. 9, 2024.
(60) Provisional application No. 63/589,289, filed on Oct. 10, 2023.

(51) Int. Cl. A61K 9/51; A61K 9/00; A61K 38/26; A61P 1/16; A61P 3/04; A61P 3/10

(52) U.S. Cl. CPC A61K 9/5153; A61K 9/0043; A61K 9/5161; A61K 38/26; A61P 1/16; A61P 3/04; A61P 3/10

(57) ABSTRACT

Methods for intranasal delivery of a GLP-1 (glucagon-like peptide-1) drug, such as tirzepatide (TZP), using a formulation of the drug encapsulated in poly(lactic-co-glycolic acid) or poly(caprolactone) nanoparticles optionally coated with chitosan and/or polyethylenimine-grafted chitosan at an optimized condition for surface charge change and particle size control are described. The disclosed intranasal formulations successfully provides dosing-dependent body fat loss in mice, showing a significant therapeutic effect.

### RELATED APPLICATIONS

This application is a continuation-in-part application of International Application No. PCT/US2024/050578, filed on Oct. 9, 2024, which claims the benefit of U.S. provisional patent application 63/589,289, filed Oct. 10, 2023, to The Trustees of Columbia University, titled “INTRANASAL DELIVERY OF TIRZEPATIDE TO TREAT OBESTIY,” the entirety of the disclosure of which is hereby incorporated by this reference.

### TECHNICAL FIELD

The subject matter disclosed herein is generally directed to intranasal delivery of an obesity drug using a formulation of the drug encapsulated in nanoparticles.

### BACKGROUND

Vast attempts have been made in the quest for treating the obesity pandemic that mainly include lifestyle modifications, surgery, and pharmacotherapy. However, the light at the end of the tunnel was not seen until the approval for GLP-1 receptor agonist medications in treating obesity. Among them, semaglutide (Wegovy®) and tirzepatide (abbreviated herein as “Tzp” or “TZP”, Zepbound®) have demonstrated significant clinical efficacy in controlling blood glucose levels as well as lowering body weight.¹ For example, a 72-week treatment of Tzp resulted in up to 22.5% weight loss in adults with obesity. GLP-1 drugs function mainly through suppressing appetite through the central nervous system, increasing insulin secretion in the pancreas, reducing glucagon production in the liver, and slowing gastric emptying in the gut, while the contribution through individual mechanisms remains to be distinguished. Although GLP-1 drugs are highly effective for weight reduction, their use is constrained by adverse effects, including gastrointestinal reactions of varying severity and risks such as thyroid tumors, acute kidney injury, acute pancreatitis, and gallbladder disease. In addition, because these agents are administered mainly by subcutaneous injection, extending the dosing interval to weekly or longer regimens has generally required higher doses and long-acting formulations. Oral administration is expected to improve patient compliance, but its development has been hindered by low bioavailability and strong ¹ For treating diabetes, semaglutide is sold under the brand names Ozempic® and Rybelsus®, and tirzepatide is sunder under the brand name Mounjaro®. gastrointestinal adverse effects. There remains a strong need for a painless, simple, and noninvasive route of self-administration to support long-term adherence to GLP-1 therapy. More importantly, organ-specific delivery may offer a promising path to reduce the adverse effects associated with systemic administration.

Tirzepatide (TZP) is a lipidated polypeptide drug developed by Eli Lilly and Company, recently approved by the US Food and Drug Administration (FDA) as an anti-diabetic and anti-obesity medication. TZP is an agonist to GIP (glucose-dependent insulinotropic polypeptide) receptor and GLP-1 (glucagon-like peptide-1) receptor, and both receptors are known to engage in stabilizing blood glucose levels and regulating food intake. Although new to the market, TZP has shown great clinical efficacy and commercial potential. Clinical observations of rebound effects following discontinuation further suggest that sustained or long-term administration may be desirable in at least some patient populations. However, TZP is currently administered via subcutaneous injection, which is unfavorable for long-term compliance. Thus, less invasive routes of administration, including intranasal delivery, are of considerable interest. However, no intranasal TZP formulation suitable for therapeutic administration has yet been established. The key challenge is that a polypeptide drug like TZP does not efficiently traverse mucosal barriers in the absence of a suitable delivery system, due at least in part to their relatively large molecular size, limited membrane permeability, and susceptibility to enzymatic and chemical degradation. Therefore, it is crucial to develop new formulations and delivery platforms capable of enhancing transport of TZP across mucosal barriers while preserving its biological activity and providing reliable therapeutic efficacy.

Citation or identification of any document in this application is not an admission that such a document is available as prior art to the present invention.

### SUMMARY

In one aspect, the present invention provides for a method of delivering a therapeutic agent having GLP-1 receptor agonist activity to a patient via intranasal administration. The method comprises intranasally administering a pharmaceutical composition comprising the therapeutic agent having GLP-1 receptor agonist activity and a nanoparticle delivery system. In certain embodiments, the therapeutic agent having GLP-1 receptor agonist activity is selected from the group consisting of tirzepatide, semaglutide, and retatrutide. In some embodiments, the therapeutic agent having GLP-1 receptor agonist activity is present in an amount effective to treat obesity, an obesity-related comorbidity, hyperglycemia, fatty liver disease, or a related metabolic disorder.

The nanoparticle delivery system encapsulates the therapeutic agent having GLP-1 receptor agonist activity. In some embodiments, the nanoparticle delivery system comprises polymeric nanoparticles comprising poly(lactic-co-glycolic acid) (PLGA) nanoparticles. In some aspects, the nanoparticle delivery system further comprises at least one positively charged biocompatible polymer, wherein the PLGA nanoparticles are coated with the at least one positively charged biocompatible polymer. In certain embodiments, the positively charged biocompatible polymer is selected from chitosan (CS) and polyethylenimine-grafted chitosan (CS-PEI). In certain embodiments, the nanoparticle delivery system comprises polymeric nanoparticles comprising PLGA optionally coated with CS-PEI. In certain embodiments, the coating-to-PLGA ratio is 0.25. In certain embodiments, the uncoated or coated nanoparticle has a polydispersity index (PdI) of 0.10±0.02 or 0.15±0.05. In certain embodiments, the coated nanoparticle has a zeta potential of 49.3±1.3 mV.

In other embodiments, the nanoparticle delivery system comprises polymeric nanoparticles comprising poly(caprolactone) (PCL) nanoparticles. In some aspects, the nanoparticle delivery system further comprises at least one positively charged biocompatible polymer, wherein the PCL nanoparticles are coated with the at least one positively charged biocompatible polymer. In certain embodiments, the positively charged biocompatible polymer is selected from chitosan (CS) and polyethylenimine-grafted chitosan (CS-PEI). In certain embodiments, the nanoparticle delivery system comprises polymeric nanoparticles comprising PCL optionally coated with CS-PEI.

In certain embodiments, the patient is being treated for obesity, an obesity-related comorbidity, hyperglycemia, fatty liver disease, or a related metabolic disorder. In certain embodiments, the therapeutic agent having GLP-1 receptor agonist activity is delivered to the olfactory bulb. In certain embodiments, the therapeutic agent having GLP-1 receptor agonist activity is delivered to one or more additional regions of the brain accessible by intranasal delivery, including, but not limited to, the olfactory tract, frontal cortex, hypothalamus, hippocampus, brainstem, or other regions of the central nervous system. In certain embodiments, the therapeutic agent having GLP-1 receptor agonist activity is administered at about 50 nmol/kg·body weight.

The foregoing and other aspects, features, and advantages will be apparent from the DETAILED DESCRIPTION, DRAWINGS, and the CLAIMS.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

An understanding of the features and advantages of the present invention will hereinafter be described in conjunction with the appended and/or included DRAWINGS, where like designations denote like elements, and:

FIG. 1—Schematic illustration of TZP-loaded nanoparticles reducing obesity.

FIG. 2A-FIG. 2F—Optimization and characterization of coated PLGA nanoparticles. (FIG. 2A-FIG. 2C) Optimization of the coating polymer (CP: CS-PEI, CS: chitosan) and coating ratio in the preparation of coated PLGA nanoparticles based on size (FIG. 2A), polydispersity index (FIG. 2B), and zeta potential (FIG. 2C); (FIG. 2D) Hydrodynamic diameter distribution of the optimized coated PLGA nanoparticle; (FIG. 2E) Representative TEM image of TZP-loaded coated PLGA nanoparticles; (FIG. 2F) Sustained release profile of the TZP-loaded PLGA nanoparticles at physiologically related in vitro conditions. Data are presented as the mean±SD.

FIG. 3A-FIG. 3C—Intranasal injections of PLGA-TZP decreased body weight and improved metabolic health. (FIG. 3A) Body weight curve and (FIG. 3B) body weight change of mice treated with or without PLGA-TZP. TZP free drug (Free), and PLGA empty treated (Veh) mice were used as the basal reference. (FIG. 3C) Body composition changes 8 days post-injection. ** p<0.01, and **** p<0.0001 for vehicle control (n=7) vs. the PLGA-TZ intranasal injection group (n=7 for PLGA-TZP group and Vehicle group, n=8 for free drug group) by 2-way ANOVA test. Data are presented as the mean±SEM.

FIG. 4—Intranasal injections of PLGA-TZP decreased body weight and improved metabolic health. Body weight change of mice treated with or without PLGA-TZP. TZP free drug (Free), and PLGA empty treated (Vehicle) mice were used as the basal reference.

FIG. 5A-FIG. 5K—Intranasal administration of GLP-1 RA nanoparticles exhibits anti-obesity effects. (FIG. 5A) Schematic diagram of the experimental design. (FIG. 5B-C) Characterization of Tzp-PLGA nanoparticles (Tzp-NP), including size distribution histograms (FIG. 5B) and release profile in PBS (FIG. 5C). (FIG. 5D and FIG. 5F) DIO C57BL/6 male mice were intranasally administered Tzp, Tzp-NP or vehicle control once daily at a dose of 100 nmol/kg. bw for 8 days. (FIG. 5D) Schematic. (FIG. 5E) Change in body weight. (FIG. 5F) Change in fat mass. (FIG. 5G) DIO male mice were administered Tzp subcutaneously (Tzp, sc, 10 nmol/kg. bw) or intranasally Tzp-NP (Tzp-NP, na, 50 nmol/kg. bw) or vehicle once daily for 3 days. (FIG. 5H and FIG. 5I) Change in body weight (FIG. 5H) and fat mass (FIG. 5I) in mice after receiving daily semaglutide subcutaneously (Sema, sc, 10 nmol/kg.bw) or intranasally of semaglutide nanoparticles (Sema-NP, 50 nmol/kg.bw) for 6 days. Water intranasally as vehicle control. (FIG. 5J and FIG. 5K) change in body weight (FIG. 5J) and fat mass (FIG. 5K) in mice after receiving daily retatrutide subcutaneously (Reta, sc, 10 nmol/kg.bw) or intranasally of retatrutide nanoparticles (Reta-NP, na, 50 nmol/kg.bw) for 7 days. Water intranasally as vehicle control. Data are mean±s.e.m. *P<0.05, **P<0.01, ***P<0.001, ****P<0.0001. Statistical significance of body weight change was assessed using 2-way ANOVA. Fat mass change was assessed using paired t-tests, with comparisons made to each mouse's baseline weight at day 0. For (B-C), n=3. For (E-F), n=7, 7, 8 (control, Tzp-NP, and Tzp group). For G, n=7 for control, and n=6 for treatment groups. For (H-I), n=6, 7, 7 (control, Sema-NP, and Sema group). For (J-K), n=8 for each group.

FIG. 6A-FIG. 6I—Intranasal delivery concentrates GLP-1 drug in the olfactory bulb and minimizes peripheral distribution. (FIG. 6A) Brain signal distribution. Mice were administered either 50 nmol/kg.bw Cy5-labeled Tzp NP or 10 nmol/kg.bw Cy5-labeled Tzp via intranasal (na) or subcutaneous (sc) routes. A control group received intranasal water. After 1 hour, in vivo imaging using an IVIS Optical Imager measured fluorescent signals. (n=3, 4, 3 for control, na, and sc groups, respectively). (FIG. 6B and FIG. 6C) Diet-induced obese (DIO) male mice fasted overnight. After treatment with Tzp NP (na), Tzp (sc), or water (vehicle control) for 1 hour, the mice were refed for 1 hour, and food intake was measured. (FIG. 6B) Schematic representation of the experimental setup. (FIG. 6C) Quantification of food intake. (n=7, 7, 6 for control, na, and sc groups, respectively). (FIG. 6D) Confocal microscopy analysis showing the distribution of Tzp-NP and Tzp in the brain 1 hour after injection. (FIG. 6E) Cy5 fluorescent signals detected in peripheral tissues 1 hour post-injection. (FIG. 6F and FIG. 6I) Mice were administered either 50 nmol/kg.bw Cy7-labeled Tzp-NP or 10 nmol/kg.bw Cy7-labeled Tzp via intranasal or subcutaneous routes. A control group received intranasal water. (n=2, 3, 3 for control, na, and sc groups, respectively). (FIG. 6F) Cy7 fluorescent signals in the brain 24 hours post-injection. (FIG. 6G) Plasma concentrations of Tzp at indicated time points (n=3, 6, 5 for control, na, and sc groups, respectively). (FIG. 6H) Cy7 fluorescent signals detected in various organs 24 hours post-injection. (FIG. 6I) Quantification of signals in different organs from panel H. The fluorescent scale bar units are photons/s/cm²/sr. Data are presented as mean±s.e.m.

FIG. 7A-FIG. 7L—The anti-obesity effect of chronic intranasal administration of Tzp-NPs. (FIG. 7A) Schematic representation of the experimental setup. Diet-induced obese (DIO) male mice received daily treatments of Tzp-NP (na), Tzp (sc), or water as control (na) for 4 weeks. Mice were sacrificed under ad libitum conditions. Food intake was measured during the first three days, and insulin tolerance tests (ITT) and glucose tolerance tests (GTT) were performed in the last two weeks. (FIG. 7B) Body weight changes during the treatment period. (FIG. 7C) Average food intake over the three-day treatment period. (FIG. 7D and FIG. 7E) Changes in fat mass (FIG. 7D) and lean mass (FIG. 7E) during the treatment. (FIG. 7F) Representative images of the mice after treatment. (FIG. 7G) Fat mass as a percentage of body weight. (FIG. 7H) Tissue weights at the time of sacrifice. (FIG. 7I) Images of iWAT) and eWAT at sacrifice after four weeks of treatment. (FIG. 7J) Histological analysis (H&E staining) of eWAT and iWAT. (FIG. 7K) Distribution of adipocyte sizes in eWAT. (FIG. 7L) Gene expression in eWAT. *p<0.05, **p<0.01, ***p<0.001, ****p<0.0001; significance was determined by 2-way ANOVA. For (C), n=7 for the vehicle group and n=6 for Tzp-treated groups. For (A-M) (except for C), n=8 for the vehicle and na groups, and n=7 for the sc group.

FIG. 8A-FIG. 8K—Intranasal delivery of Tzp-NP improves obesity-associated metabolic disorders similarly to subcutaneous administration. (FIG. 8A-FIG. 8H) The experimental design is the same as described in FIG. 3, panel A. (FIG. 8A and FIG. 8B) Glucose tolerance test (GTT) (FIG. 8A) and insulin tolerance test (ITT) (FIG. 8B) performed after 2.5 and 2 weeks of Tzp treatment, respectively. (FIG. 8C-FIG. 8E) Plasma levels of insulin (FIG. 8C), non-esterified fatty acids (NEFA) (FIG. 8D), and triglycerides (TG) (FIG. 8E). (FIG. 8F) Histological analysis (H&E staining) of liver tissue. (FIG. 8G) Plasma alanine aminotransferase (ALT) levels. (FIG. 8H) Gene expression analysis in the liver. (FIG. 8I and FIG. 8J) Blood glucose and insulin levels in mice 1 hour after injection of Tzp-NP (na), Tzp (sc), or water as control. (FIG. 8K) Time course of glucose changes in mice receiving Tzp-NP or Tzp. Data are presented as mean±s.e.m. For (A-H), n=8 for the vehicle and intranasal groups; n=7 for the subcutaneous group. For (I, J), n=7, 6, 7 for the vehicle, intranasal, and subcutaneous groups, respectively. For (K), n=6 for each group. Statistical significance was determined using two-way ANOVA.

FIG. 9A-FIG. 9F—Characterization of TZP-PLGA nanoparticles includes (FIG. 9A) a transmission electron microscopy (TEM) image, (FIG. 9B) hydrodynamic diameter, (FIG. 9C) polydispersity index (PDI), and (FIG. 9D) zeta potential. (FIG. 9E) Body weight changes in diet-induced obese (DIO) female mice after a single intranasal injection of Tzp-NP or water (control). (FIG. 9F) Body weight changes in DIO male mice after receiving one dose of freshly prepared Tzp-NP or Tzp-NP stored at 4° C. for 3 or 7 days. For (B-D), n=3 for each group. For (E-F), n=5 for each group. Statistical significance was determined using a t-test or two-way ANOVA.

The figures herein are for illustrative purposes only and are not necessarily drawn to scale.

### DETAILED DESCRIPTION

Detailed aspects and applications of the disclosure are described below in the following drawings and detailed description of the technology. Unless specifically noted, it is intended that the words and phrases in the specification and the claims be given their plain, ordinary, and accustomed meaning to those of ordinary skill in the applicable arts.

In the following description, and for the purposes of explanation, numerous specific details are set forth in order to provide a thorough understanding of the various aspects of the disclosure. It will be understood, however, by those skilled in the relevant arts, that embodiments of the technology disclosed herein may be practiced without these specific details. It should be noted that there are many different and alternative configurations, devices and technologies to which the disclosed technologies may be applied. The full scope of the technology disclosed herein is not limited to the examples that are described below.

The singular forms “a,” “an,” and “the” include plural referents unless the context clearly dictates otherwise. Thus, for example, reference to “a step” includes reference to one or more of such steps.

The words “exemplary,” “example,” or various forms thereof are used herein to mean serving as an example, instance, or illustration. Any aspect or design described herein as “exemplary” or as an “example” is not necessarily to be construed as preferred or advantageous over other aspects or designs. Furthermore, examples are provided solely for purposes of clarity and understanding and are not meant to limit or restrict the disclosed subject matter or relevant portions of this disclosure in any manner. It is to be appreciated that a myriad of additional or alternate examples of varying scope could have been presented but have been omitted for purposes of brevity.

When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable. The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

Throughout the description and claims of this specification, the words “comprise” and “contain” and variations of the words, for example “comprising” and “comprises”, mean “including but not limited to”, and are not intended to (and do not) exclude other components.

As required, detailed embodiments of the present disclosure are included herein. It is to be understood that the disclosed embodiments are merely exemplary of the invention that may be embodied in various forms. Therefore, specific structural and functional details disclosed herein are not to be interpreted as limits, but merely as a basis for teaching one skilled in the art to employ the present invention. The specific examples below will enable the disclosure to be better understood. However, they are given merely by way of guidance and do not imply any limitation.

The present disclosure may be understood more readily by reference to the following detailed description taken in connection with the accompanying figures and examples, which form a part of this disclosure. It is to be understood that this disclosure is not limited to the specific materials, devices, methods, applications, conditions, or parameters described and/or shown herein, and that the terminology used herein is for the purpose of describing particular embodiments by way of example only and is not intended to be limiting of the claimed inventions. The term “plurality”, as used herein, means more than one.

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N. Y. 1994), March, Advanced Organic Chemistry Reactions, Mechanisms and Structure 4th ed., John Wiley & Sons (New York, N. Y. 1992); and Marten H. Hofker and Jan van Deursen, Transgenic Mouse Methods and Protocols, 2^{nd} edition (2011).

The term “optional” or “optionally” means that the subsequent described event, circumstance or substituent may or may not occur, and that the description includes instances where the event or circumstance occurs and instances where it does not.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily—but may be—all referring to the same embodiment. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### Overview

GLP-1 class of drugs, which are therapeutic agents having GLP-1 receptor agonist activity, are blockbusters in treating obesity. However, the current subcutaneous injection creates a series of problems that decrease compliance and increase risks. Oral delivery is either at extremely low bioavailability or otherwise may not work well. The intranasal delivery of TZP reported herein offers a novel, more convenient, and potentially safer approach to maximize its market.

In contrast to the regular routes of administration, intranasal delivery provides unique advantages. First, the nasal mucosal environment is less acidic, less digestive, and simpler to navigate than the oral delivery route. Second, due to the high permeability and the large surface area of intranasal vasculatures, intranasal delivery offers superior bioavailability and faster absorption into the bloodstream, almost comparable with intravenous injection. Most notably, intranasal administration permits drugs to bypass the blood-brain barrier via the olfactory nasal neuroepithelium, as evidenced by the approval of Esketamine, an antidepressant known to exert its therapeutic effects in the brain, and Desmopressin to treat central cranial diabetes insipidus. Indeed, peptide drugs, including insulin, glucagon, and GLP-1 drugs have been investigated for intranasal delivery due to these advantages, particularly in the context of type 2 diabetes. However, intranasal delivery of recombinant human GLP-1 successfully reduced blood glucose levels in type 2 diabetes patients, but without reducing body weight, total food intake, or hunger sensation (Ueno H, Mizuta M, Shiiya T, et al. Exploratory trial of intranasal administration of glucagon-like peptide-1 in Japanese patients with type 2 diabetes. Diabetes Care. 2014;37(7):2024-2027). Another study in obese adults intranasally administered peptide YY3-36 (PYY3-36) for 12 weeks did not result in body weight loss either (Gantz I, Erondu N, Mallick M, et al. Efficacy and safety of intranasal peptide YY3-36 for weight reduction in obese adults. J Clin Endocrinol Metab. 2007;92(5):1754-1757). Therefore, there are critical gaps to liberate the therapeutic potential of GLP-1 drugs in obesity management through this intranasal delivery route with unique advantages.

Described herein is an intranasal nanoparticle system for delivering GLP-1 drugs into the brain. With a more restricted delivery to the brain but minimal peripheral distribution, Applicants achieved a prevailing anti-obesity efficacy for GLP-1 drugs including semaglutide, Tzp, and the triple retatrutide. This restricted delivery reveals an intriguing mechanism of GLP-1 RA to suppress appetite ignited in the olfactory bulb, which is distinct from the regular systemic administration. Besides weight control, the metabolic benefits on improving insulin sensitivity and glucose and lipid metabolism are comparable to systemic subcutaneous administration. More importantly, by avoiding distribution to the hindbrain, intranasal delivery of tirzepatide alleviated the gastrointestinal adverse events in rats and minks. Therefore, intranasal delivery offers a promising approach for maximizing the therapeutic effects GLP-1 drugs with potentially increased compliance and safety.

Applicants first designed and developed a nanoparticle delivery system that can encapsulate TZP efficiently and deliver TZP effectively via intranasal administration. In one implementation, nanoparticle delivery system utilizes poly(lactic-co-glycolic acid) (PLGA). PLGA, or poly(lactic-co-glycolic acid), is a series of biodegradable and biocompatible polymers approved by the FDA for therapeutic usage (see, e.g., Danhier F, Ansorena E, Silva J M, Coco R, Le Breton A, Préat V. PLGA-based nanoparticles: an overview of biomedical applications. J Control Release. 2012;161(2):505-522). It is also highly flexible for fabrication and manufacturing, enabling the formation of both macroscopic medical implants and drug-encapsulating nanoparticles. The degradability of PLGA can be tuned by adjusting the ratios between the two monomers, lactic acid and glycolic acid. It is a safe candidate for various drug delivery methods.

In some aspects, the PLGA is coated with at least one positively charged biocompatible polymer. To enhance the cross-mucosa transportation, Applicants developed a series of new carriers by coating the PLGA nanoparticle with chitosan (CS) and chitosan derivatives, polyethylenimine-grafted chitosan (CS-PEI). Chitosan is a natural polysaccharide derived from chitin, which is a common component of the shells of crustaceans (e.g., crabs and lobsters). It is highly biocompatible and biodegradable. Chitosan has been proven to assist nanoparticle transportation across mucosa, as its amino groups, when protonated in water, carry positive charges and interact with the negatively charged mucus layer. The PEI grafting gives CS-PEI a higher positive charge density than chitosan, which improves transportation, and a higher solubility in water, which helps the colloidal stability of the resulting nanoparticles. The experiments on obese mice have shown that with the assistance of the nanoparticle, intranasal delivery can result in a promising therapeutic effect of TZP that is comparable with subcutaneous injection, which is unachievable with intranasal dosing of the free drug form alone.

Polymers in the same general class of biodegradable polymers as PLGA and having similar functions in drug delivery are suitable alternatives to PLGA. For example, poly(caprolactone) (PCL) belongs to the same general class of biodegradable polymers as PLGA and possesses similar drug delivery functions as PLGA. Furthermore, PCL has been demonstrated to successfully facilitate intranasal administration of therapeutic agents. Thus, in another implementation, nanoparticle delivery system comprises uncoated PCL or PCL coated with at least one positively charged biocompatible polymer. In particular embodiments, the nanoparticle delivery system comprises PCL coated with CS or CS-PEI.

Whether GLP-1 receptor agonists (RAs) function through the central and peripheral systems to control body weight and improve metabolic functions remains unclear, mainly limited by the lack of efficient delivery to the brain. Here, Applicants show that nanomedicine-mediated intranasal delivery of tirzepatide to the brain achieves comparable anti-obesity effect and metabolic improvements to subcutaneous administration, despite minimal distributions in the circulation and peripheral tissues. Tirzepatide was detected primarily at the olfactory bulb region without reaching hypothalamus in acute intranasal administration; however, food intake and hyperglycemia were already suppressed. Three-dimensional whole-brain c-Fos imaging revealed the majority overlapped brain regions of neuron activation between the intranasal and subcutaneous delivery routes with some distinct areas. Notably, by avoiding distribution to the hindbrain (brainstem), intranasal delivery of tirzepatide appears to alleviate the gastrointestinal adverse events in rats and minks. These findings suggest that intranasal delivery of GLP-1 drugs potentially offer a safer and more patient-friendly therapeutic option for obesity and diabetes management.

### Terminology and Definitions

The terms “therapeutic agent”, “therapeutic capable agent” or “treatment agent” are used interchangeably and refer to a molecule or compound that confers some beneficial effect upon administration to a subject. The beneficial effect includes enablement of diagnostic determinations; amelioration of a disease, symptom, disorder, or pathological condition; reducing or preventing the onset of a disease, symptom, disorder or condition; and generally counteracting a disease, symptom, disorder or pathological condition.

As used herein, “treatment” or “treating,” or “palliating” or “ameliorating” are used interchangeably. These terms refer to an approach for obtaining beneficial or desired results including but not limited to a therapeutic benefit and/or a prophylactic benefit. By therapeutic benefit is meant any therapeutically relevant improvement in or effect on one or more diseases, conditions, or symptoms under treatment. For prophylactic benefit, the compositions may be administered to a subject at risk of developing a particular disease, condition, or symptom, or to a subject reporting one or more of the physiological symptoms of a disease, even though the disease, condition, or symptom may not have yet been manifested. As used herein “treating” includes ameliorating, curing, preventing it from becoming worse, slowing the rate of progression, or preventing the disorder from re-occurring (i.e., to prevent a relapse).

The term “effective amount” or “therapeutically effective amount” refers to the amount of an agent that is sufficient to effect beneficial or desired results. The therapeutically effective amount may vary depending upon one or more of: the subject and disease condition being treated, the weight and age of the subject, the severity of the disease condition, the manner of administration and the like, which can readily be determined by one of ordinary skill in the art. The term also applies to a dose that will provide an image for detection by any one of the imaging methods described herein. The specific dose may vary depending on one or more of: the particular agent chosen, the dosing regimen to be followed, whether it is administered in combination with other compounds, timing of administration, the tissue to be imaged, and the physical delivery system in which it is carried.

A “pharmaceutical composition” refers to a composition that usually contains an excipient, such as a pharmaceutically acceptable carrier that is conventional in the art and that is suitable for administration to cells or to a subject.

As used herein “biocompatible” refers to compatibility with living tissue or a living system by not being toxic, injurious, or physiologically reactive and not causing immunological rejection.

### Methods of Treating Obesity and Obesity Comorbidities

In example embodiments, subjects are treated intranasally with a pharmaceutical composition or therapeutic agent comprising a GLP-1 drug. In example embodiments, the pharmaceutical composition is administered in the form of a nanoparticle coated with positively charged polymers.

### Patients and Diseases or Conditions

In example embodiments, the compositions disclosed herein are used to treat subjects. The treatments may be prophylactic. In example embodiments, the compositions disclosed herein can be used for treating subjects having any condition or disease related to obesity, including without limitation obesity comorbidities such as diabetes and liver steatosis, or any other condition causing or associated with high blood glucose levels and/or fatty liver. Non-limiting conditions or diseases include type 2 diabetes, atherosclerotic cardiovascular disease, cardiovascular disease, obesity, non-alcoholic fatty liver disease, metabolic liver disease, polycystic ovary syndrome, diseases of the reward system (e.g., addictions, binge eating disorder, or substance use disorder), glucose intolerance, insulin resistance, and depression (e.g., GLP-1 agonists have shown antidepressant and neuroprotective effects). In example embodiments, subjects at risk for obesity associated cancers are treated (e.g., esophageal, colorectal, endometrial, gallbladder, kidney, liver, ovarian, and pancreatic cancer as well as meningioma and multiple myeloma). In example embodiments, any subject having a class of obesity is treated. For example, subjects having class I obesity—overweight (BMI 25.0-29.9 kg/m²), class II obesity—obesity (BMI 30.0-39.9 kg/m²), or class III obesity—extreme obesity (BMI>40 kg/m2) are treated. In example embodiments, subjects with a BMI over 30 or a BMI over 27 with at least one weight-related comorbidity are treated with the compositions described herein. In example embodiments, the compositions disclosed herein are used for cosmetic weight loss.

In example embodiments, the GLP-1 drug is administered at about 50 nmol/kg·body weight. For example, 1, 5, 10, 15, 20, 25, 30, 35, 40, 45, 50 nmol/kg·body weight, or an effective amount or therapeutically effective amount determined by a physician and the disease or condition being treated (e.g., the patient is administered an amount where they are no longer obese). In example embodiments, humans are more sensitive to GLP-1 drugs than mice, such that the dose is lower to treat human patients than mice. In example embodiments, rather than one injection/weeks, intranasal administration can be more frequent (e.g., before each meal, daily, every other day). An advantage of more frequent administration is that the treatment can more closely mimick physiological fluctuations as compared to systemic injection.

### GLP-1 Receptor Agonists

As used herein “GLP-1 drugs” refers to glucagon-like peptide-1 (GLP-1) receptor agonists, also known as GLP-1 analogs, GLP-1DAs or incretin mimetics. Glucagon-like peptide-1 (GLP-1) receptor agonists also refers to a class of anorectic drugs that reduce blood sugar and energy intake by activating the GLP-1 receptor. They mimic the actions of the endogenous incretin hormone GLP-1 that is released by the gut after eating. GLP-1 agonists were initially developed for type 2 diabetes. The 2022 American Diabetes Association standards of medical care recommend GLP-1 agonists as a first line therapy for type 2 diabetes, specifically in patients with atherosclerotic cardiovascular disease or obesity. The drugs were also noted to reduce food intake and body weight significantly, and some have also been approved to treat obesity in the absence of diabetes. They are also in development for other indications, such as non-alcoholic fatty liver disease, polycystic ovary syndrome, and diseases of the reward system such as addictions. GLP-1 agonists work by activating the GLP-1 receptor. They slow gastric emptying, inhibit the release of glucagon, and stimulate insulin production, therefore reducing hyperglycemia in people with type 2 diabetes. They also reduce food intake and therefore body weight, making them an effective treatment for obesity. Some of the metabolic effects of GLP-1 agonists in rodents are mediated via increased synthesis of fibroblast growth factor 21 (FGF21). Dual GLP-1/FGF21 receptor agonists have been developed by pharmaceutical companies. In example embodiments, the compositions disclosed herein include both GLP-1/FGF21 receptor agonists.

Non-limiting GLP-1 drugs that can be incorporated into the nanoparticles described herein include exenatide (brand names Byetta® and Bydureon™, manufactured by AstraZeneca), approved in 2005/2012, liraglutide (Victoza® for diabetes, Saxenda® for obesity, manufactured by Novo Nordisk), approved in 2010, albiglutide (Tanzeum™, manufactured by GSK), approved in 2014, dulaglutide (Trulicity®, manufactured by Eli Lilly), approved in 2014, lixisenatide (Lyxumia™ in Europe, Adlyxin™ in the United States, manufactured by Sanofi), approved in 2016, semaglutide (Ozempic® and Rybelsus® for diabetes, Wegovy® for obesity, manufactured by Novo Nordisk), approved in 2017, tirzepatide (TZP; dual GLP-1 and GIP agonist; Mounjaro® for diabetes, Zepbound® for obesity, manufactured by Eli Lilly), approved in 2022, dulaglutide (Trulicity®), exenatide (Byetta®), exenatide extended-release (Bydureon®), Orforglipron™ (a nonpeptide oral GLP-1 receptor agonist), Efocipegtrutide™ (HM15211) (see, e.g., Abdelmalek M F, Suzuki A, Sanchez W, et al. A phase 2, adaptive randomized, double-blind, placebo-controlled, multicenter, 52-week study of HM15211 in patients with biopsy-confirmed non-alcoholic steatohepatitis—Study design and rationale of HM-TRIA-201 study. Contemp Clin Trials. 2023;130:107176), and retatrutide (RETA; LY3437943) (see, e.g., Coskun T, Urva S, Roell WC, et al. LY3437943, a novel triple glucagon, GIP, and GLP-1 receptor agonist for glycemic control and weight loss: From discovery to clinical proof of concept. Cell Metab. 2022;34(9):1234-1247.e9; and Jastreboff A M, Kaplan L M, Frías J P, et al. Triple-Hormone-Receptor Agonist Retatrutide for Obesity—A Phase 2 Trial. N Engl J Med. 2023;389(6):514-526).

### Nanoparticles

In example embodiments, a GLP-1 drug is encapsulated in nanoparticles. In example embodiments, the nanoparticles are PLGA or PCL. In example embodiments, the PLGA nanoparticles are about 50 nm, 60 nm, 70 nm, 80 nm, 90 nm, 100 nm, 110, nm, 120 nm, or 130 nm, preferably about 100 nm. In example embodiments, the nanoparticles are functionalized with one or more positively charged polymers (i.e., coated). In example embodiments, the nanoparticles are generated followed by adding positively charged polymers that coat the nanoparticles based on the electrostatic interaction between the negatively charged surface of the naked nanoparticles and the positively charged polymers. In preferred embodiments, the one or more positively charged polymers are selected from CS and CS-PEI coatings. In example embodiments, CS and/or CS-PEI coatings are used at various ratios to PLGA to obtain the desired nanoparticles. In example embodiments, CS-PEI coating is used.

In example embodiments, the coating-to-nanoparticle ratio is 0.10 to 0.40. In some implementations, the coating-to-PLGA ratio is at 0.10, 0.15, 0.20, 0.25, 0.30, 0.35, or 0.40. In certain implementations, the coating-to-PLGA ratio is preferably at 0.25.

In example embodiments, the coated nanoparticle has a hydrodynamic diameter of about 110 nm, 115 nm, 120 nm, 125 nm, or 130 nm, preferably, about 120.5±1.2 nm. In example embodiments, the coated nanoparticle has a PdI of 0.1±0.02 and a zeta potential of 49.3±1.3 mV. In other example embodiments, the coated nanoparticle has a PdI of 0.15±0.05 and a zeta potential of 49.3±1.3 mV.

### Generating Nanoparticles

In example embodiments, PLGA or PCL nanoparticles are generated by any method known in the art. In example embodiments, PLGA nanoparticles are generated by emulsification-evaporation. This technique allows encapsulation of both hydrophobic and hydrophilic drugs on the micro-or nanoscale. In example embodiments, PLGA is dissolved into an organic phase (oil) that is emulsified with a surfactant or stabilizer in an oil immiscible phase (usually water). Hydrophobic drugs are added directly to the oil phase, whereas hydrophilic drugs may be first emulsified with the polymer solution prior to particle formation. High intensity sonication bursts can be employed to facilitate formation of small polymer droplets. The resulting emulsion is added to a larger aqueous phase and stirred for several hours, allowing the solvent to evaporate. The polymer precipitates as the solvent is removed, and hardened nanoparticles are collected and washed by centrifugation prior to lyophilization and long-term storage. In example embodiments, PLGA are generated using emulsion-based techniques followed by adding positively charged polymers that coat the PLGA nanoparticles based on the electrostatic interaction between the negatively charged surface of the naked PLGA nanoparticles and the positively charged polymers.

In example embodiments, PLGA nanoparticles are generated by nanoprecipitation (see, e.g., Salatin S, Barar J, Barzegar-Jalali M, Adibkia K, Kiafar F, Jelvehgari M. Development of a nanoprecipitation method for the entrapment of a very water-soluble drug into Eudragit RL nanoparticles. Res Pharm Sci. 2017;12(1):1-14). In this approach, the polymer and the drug are dissolved in an organic solvent (such as acetone or DMSO) and then added dropwise to water. The organic solvent is evaporated, and the particles are collected as a pellet using centrifugation. In example embodiments, nanoparticles are generated by Flash NanoPrecipitation (FNP) (see, e.g., Markwalter C E, Pagels R F, Wilson B K, Ristroph K D, Prud'homme R K. Flash NanoPrecipitation for the Encapsulation of Hydrophobic and Hydrophilic Compounds in Polymeric Nanoparticles. J Vis Exp. 2019;(143):10.3791/58757).

In example embodiments, PLGA nanoparticles are generated by microfluidics-assisted synthesis (see, e.g., Hung L H, Lee A P. Microfluidic devices for the synthesis of nanoparticles and biomaterials. J Med Biol Eng. 2007;27(1): 1-6; and Lababidi N, Sigal V, Koenneke A, Schwarzkopf K, Manz A, Schneider M. Microfluidics as tool to prepare size-tunable PLGA nanoparticles with high curcumin encapsulation for efficient mucus penetration. Beilstein J Nanotechnol. 2019;10:2280-2293).

Further embodiments are illustrated in the following Examples which are given for illustrative purposes only and are not intended to limit the scope of the invention.

### EXAMPLES

### Example 1—Tzp-loaded Nanoparticles for Intranasal Delivery

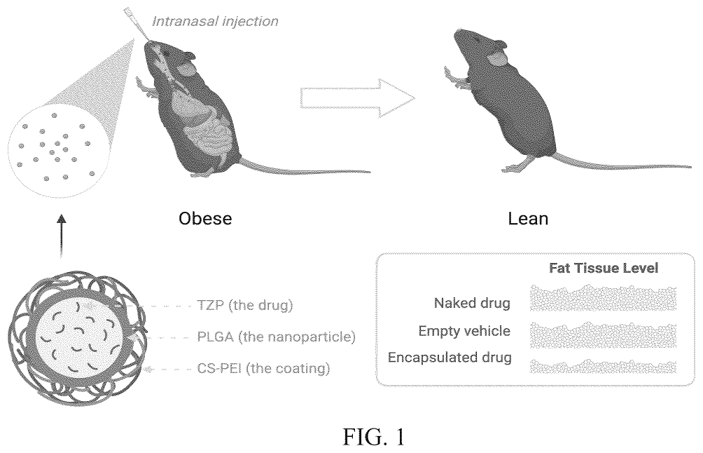

This invention is to report TZP-loaded nanoparticles for intranasal delivery with promising anti-obesity effects. Applicants encapsulated TZP in PLGA nanoparticles and coated them with CS and CS-PEI at an optimized condition for surface charge change and particle size control. The resulting formulation was tested on obese mice. Consistent dosing-dependent body fat loss has been observed, showing a significant therapeutic effect (FIG. 1).

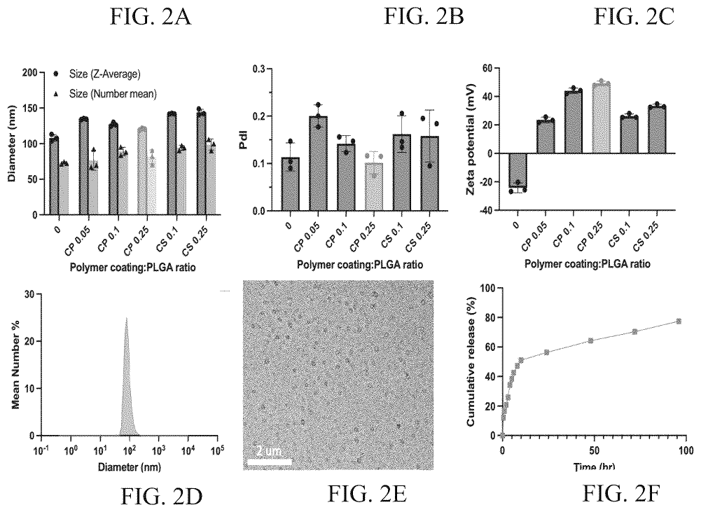

Here is a brief description of the development of the nanoparticles. Applicants first generated the PLGA nanoparticles using emulsion-based techniques. Then, in order to functionalize the nanoparticles with polymers, Applicants tested both CS and CS-PEI coatings at various ratios to PLGA and characterized the self-assembly behaviors of the resulting nanoparticles, including hydrodynamic size, polydispersity index (PdI), and zeta potential (FIG. 2A-FIG. 2F). Both coatings relied on the electrostatic interaction between the negatively charged surface of the naked PLGA nanoparticles and the positively charged polymers. Due to the PEI grafting, CS-PEI had a higher density of positive charges than CS. Consequently, CS-PEI coated PLGA nanoparticles not only had a higher surface charge, but also formed a more compact coating, which explains that at the same coating ratio, CS-PEI coated nanoparticles had a smaller size than the CS coated nanoparticles. Given that the nanoparticle size before coating was already 100 nm, a minimal increase in size after coating is desired for efficient intranasal drug delivery. Therefore, the formulation of CS-PEI coating with the coating-to-PLGA ratio at 0.25 was optimal. Moreover, this formulation also presented the lowest PdI values, which means the most uniform and consistent distribution of nanoparticle sizes. Therefore, it was the finally optimized formulation with a hydrodynamic diameter of 120.5±1.2 nm, a PdI of 0.1±0.02, and a zeta potential of 49.3±1.3 mV. In addition, the sustained release of TZP was also proven by an in vitro experiment with physiologically related conditions at 37° C. in phosphate buffer solutions. The release curve showed that only 50% of the loaded drug was released in the first 10 hr. At 48 hr, the release just reached 60 %, and then slowly climbed to 80% at 96 hr. These data showed good potential of an intranasal nanocarrier for TZP with attractive physical characteristics and controlled release behavior.

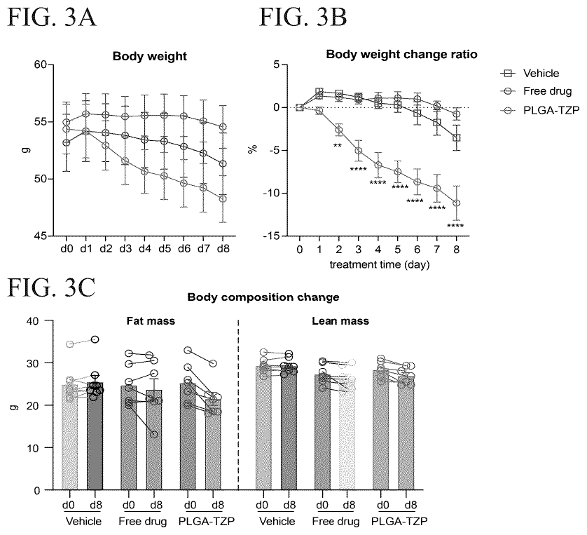

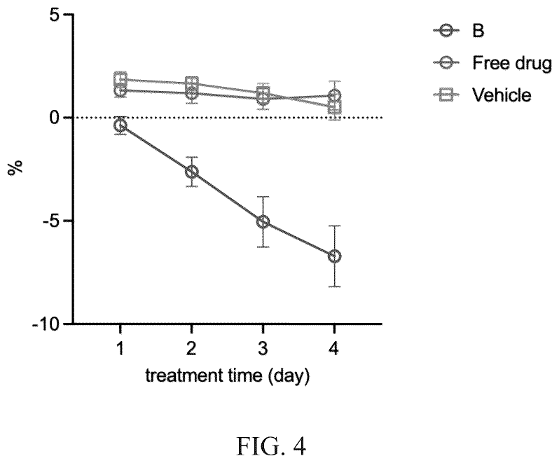

Next, Applicants investigated whether PLGA-TZP nanoparticles have the effect on body weight loss. To initiate obesity in C57BL/6 mice, Applicants subjected them to a high-fat diet (HFD). Following obesity induction, the obese mice underwent daily intranasal injections of PLGA-TZP at a dosage of 100 nmol/kg body weight, and their body weight changes were closely monitored. Over the course of a week, PLGA-TZP led to a gradual reduction in body weight, with a daily decrease of approximately 2% (FIG. 3A and FIG. 3B). After one week of treatment, the PLGA-TZP group exhibited a weight reduction of around 10% compared to both the vehicle and TZP-free drug groups. Analysis of body composition (MRI) revealed that the weight loss primarily stemmed from a decrease in fat mass (FIG. 3C).

Subsequent to these findings, Applicants can investigate the metabolic advantages associated with PLGA-TZP. Applicants expect to observe that PLGA-TZP treatment ameliorates glucose intolerance and insulin resistance that associate with obesity.

The limitations in the application of TZP in weight loss stem from their associated side effects. Among these, pancreatitis, gastrointestinal adverse events, and injection site inflammation are frequently reported with TZP. The controlled and constant release by PLGA-TZP is predicted to alter the pharmacodynamics (PD) of TZP in vivo, therefore avoiding acute high concentration by the current S. C. injection. In an effort to assess the potential mitigation of TZP side effects through intranasal administration of PLGA-TZP compared to conventional subcutaneous (S. C.) injection, Applicants can conduct a comparative analysis. This comparison can involve evaluating the side effects of TZP when administered via intranasal injection versus S. C. injection, with the goal of achieving equivalent levels of body weight loss efficacy in both groups. Applicants expect that the intranasal administration of PLGA-TZP will present advantages in sidestepping side effects. Applicants can also evaluate TZP's effects in the bone and other organs.

In summary, this non-invasive intranasal administration of PLGA-TZP not only sustains the benefits of weight loss and enhances metabolic health, similar to S. C. TZP injection, but more importantly, it improves patient compliance and circumvents side effects.

### Example 2—Developing GLP-1 RA Nanoparticles for Intranasal Delivery to Reduce Body Weight

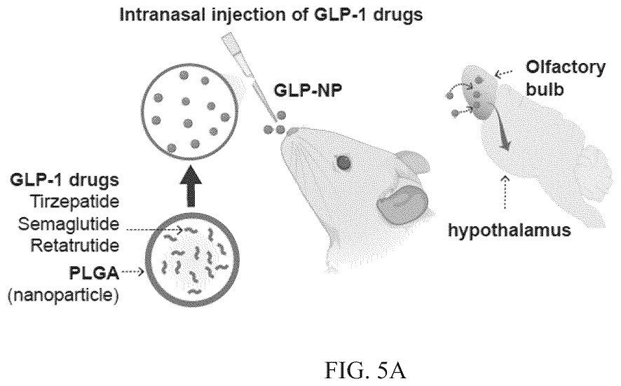

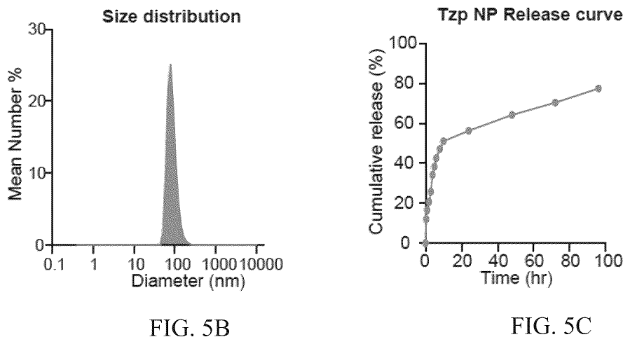

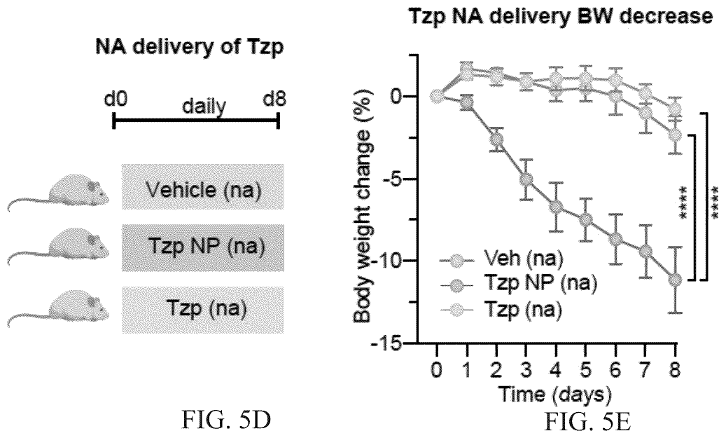

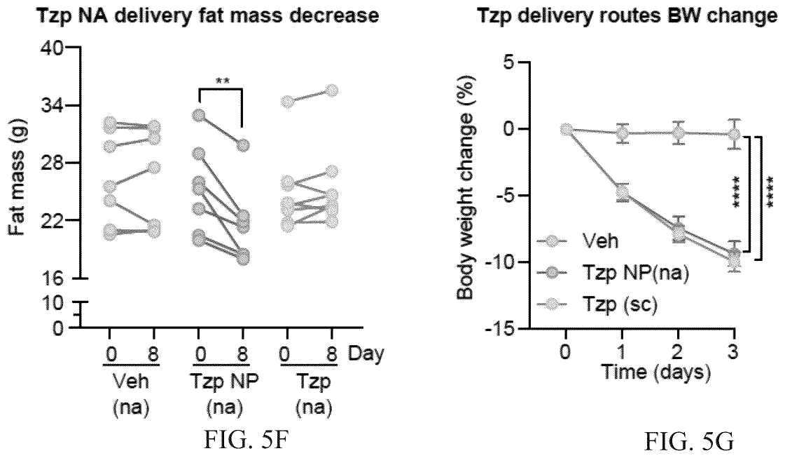

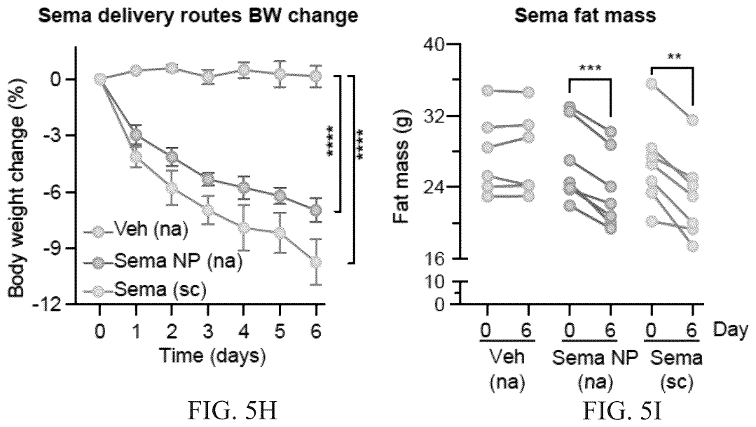

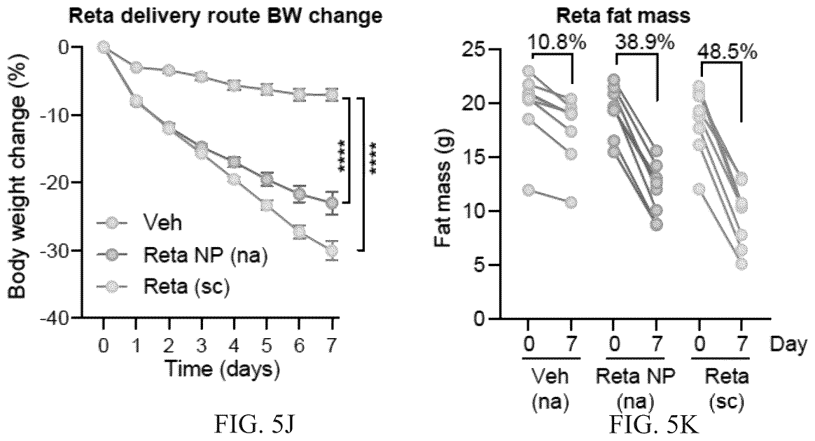

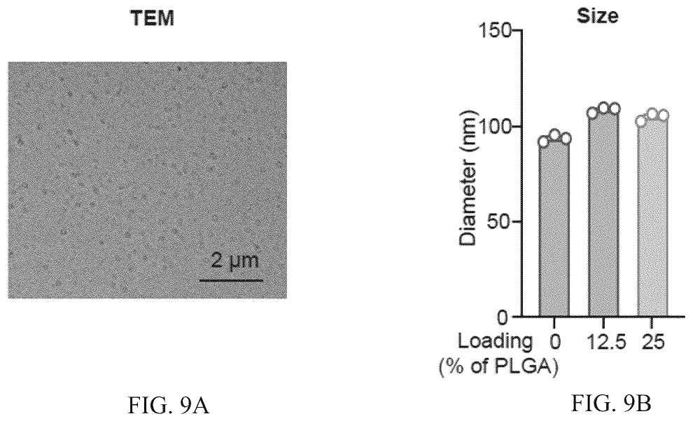

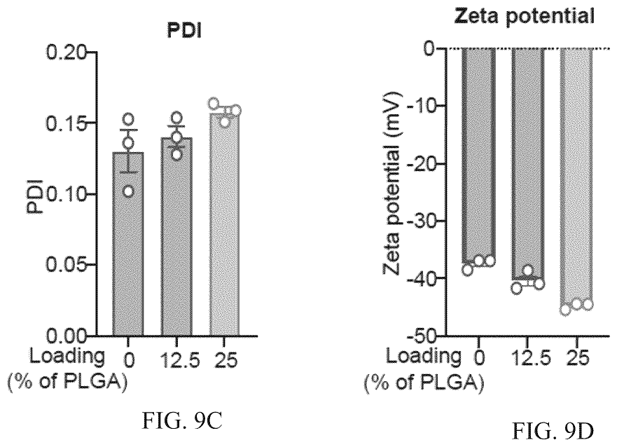

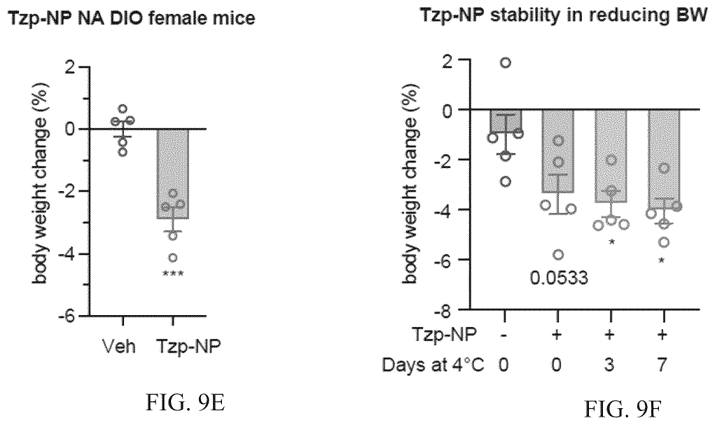

To investigate whether brain administration of GLP-1 drugs is applicable for weight control, Applicants gave diet-induced obese (DIO) mice Tzp via daily intranasal injection but failed to reduce body weight, suggesting ineffectiveness in traversing the barriers to function in the brain. To address this limitation, Applicants employed a biodegradable and biocompatible polymer PLGA (poly(lactic-co-glycolic acid)), which is an FDA-approved drug carrier, to encapsulate Tzp, creating nanoparticles (Tzp-NPs) to facilitate intranasal delivery (FIG. 5A). Tzp-NPs were prepared using emulsion-based techniques, and the optimized formulation was characterized with a hydrodynamic diameter of 104.7±2.1 nm, a polydispersity index (PdI) of 0.16±0.01, and a zeta potential of −44.8±0.6 mV (FIG. 5B and FIG. 9A-FIG. 9D). Tzp-NPs also showed sustained release of Tzp, reaching 50% at 10 hours and slowly climbing to 80% at 96 hours (FIG. 5C).

Applicants then treated DIO mice with Tzp-NPs through daily intranasal injections at a dosage of 100 nmol/kg·BW (FIG. 5D). These NP carriers enabled efficient diffusion of Tzp across the mucosal layer and the blood brain barrier (BBB), resulting in a weight reduction at 12% in an 8-day treatment, in striking contrast to the blunted effect of free Tzp (FIG. 5E). The loss of body weight is primary contributed by the reduction of fat mass (FIG. 5F) and was reproduced in female DIO mice, showing 3% weight loss in 24 hours (FIG. 9E). Moreover, Tzp-NPs displayed good stability as they reduced the same BW after being dissolved for up to 1 week at 4° C. (FIG. 9F).

Next, Applicants compared the anti-obesity efficacy of intranasal delivery to the regular systemic delivery. In DIO mice, intranasal administration of a dose of 50 nmol/kg body weightTzp-NP achieved a comparable 10% weight reduction to subcutaneous injection of free Tzp (10 nmol/kg·bw) within a 3-day treatment (FIG. 5G). Furthermore, Applicants assessed whether PLGA NP encapsulation is a general approach for intranasal delivery of GLP-1 RAs. Six-day intranasal administration of semaglutide (sema) NPs reduced 8% BW in DIO mice, comparing to 10% of subcutaneous injection of free semaglutide (FIG. 5H), underlain by the same fat mass reduction (FIG. 5I). Similarly, for the triple GLP-1 drug retatrutide, intranasal delivery of its NPs caused a 22% weight loss and 38.9% fat loss in one week, less potent than the subcutaneous injection (FIG. 5J and FIG. 5K). Collectively, intranasal delivery of NP-encapsulated GLP-1 drugs appears a feasible therapy for treating obesity in a DIO mouse model.

### Example 3—Intranasal Delivery Concentrates GLP-1 Drug in the Olfactory Bulb and Minimizes Peripheral Distribution

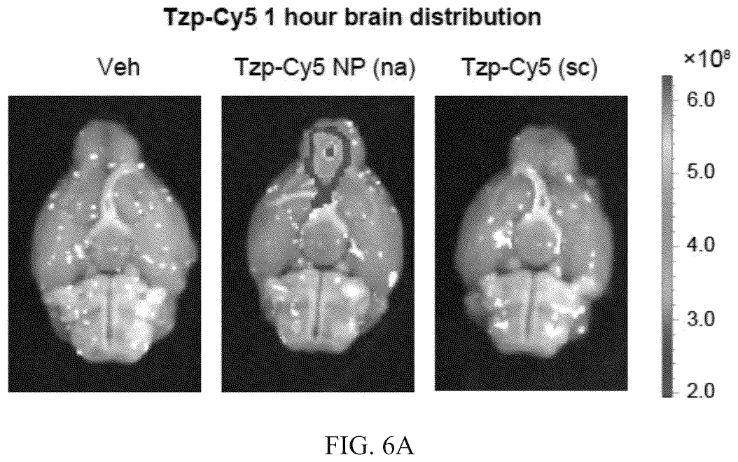

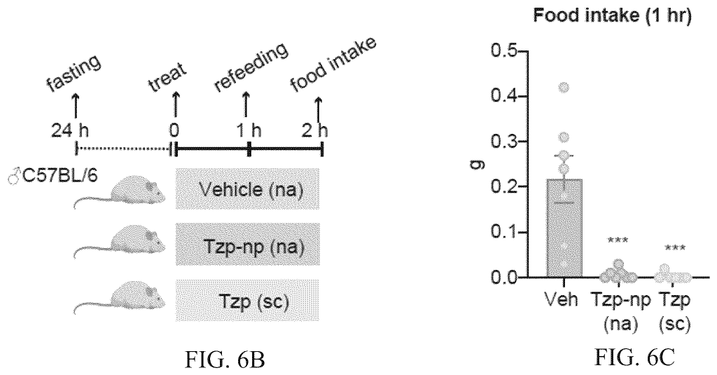

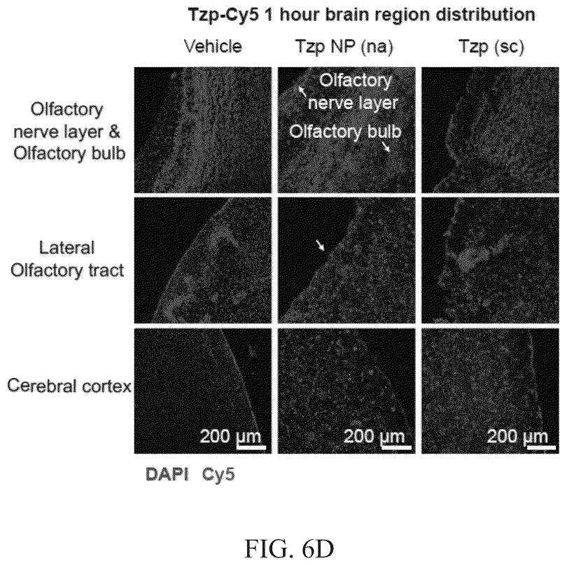

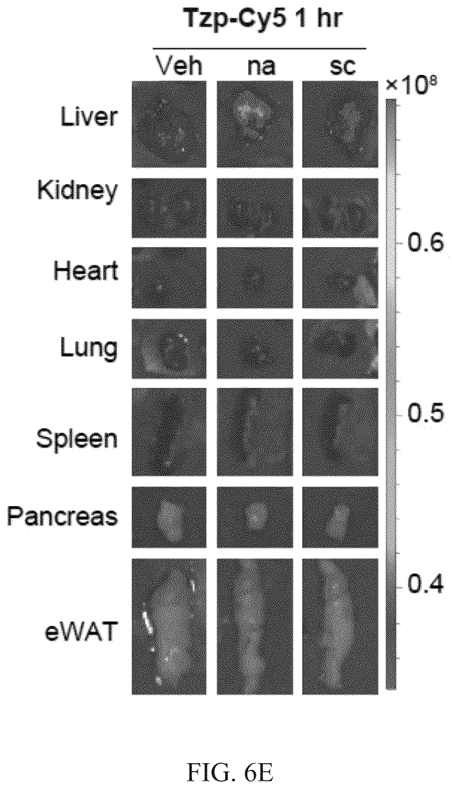

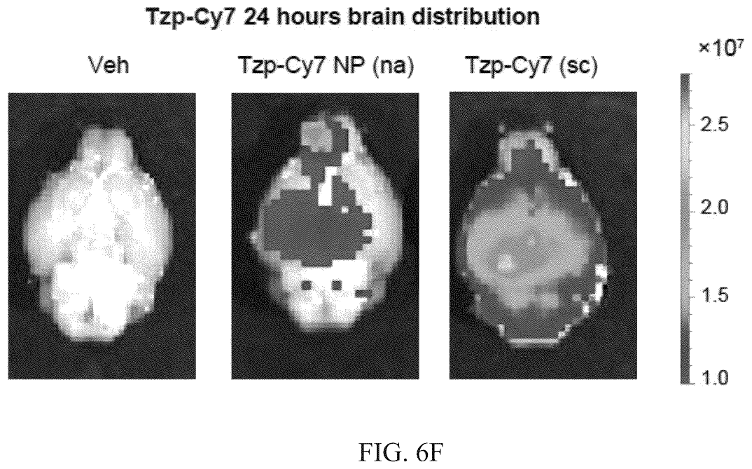

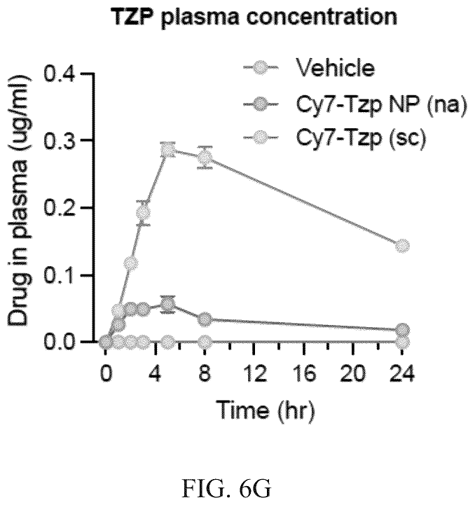

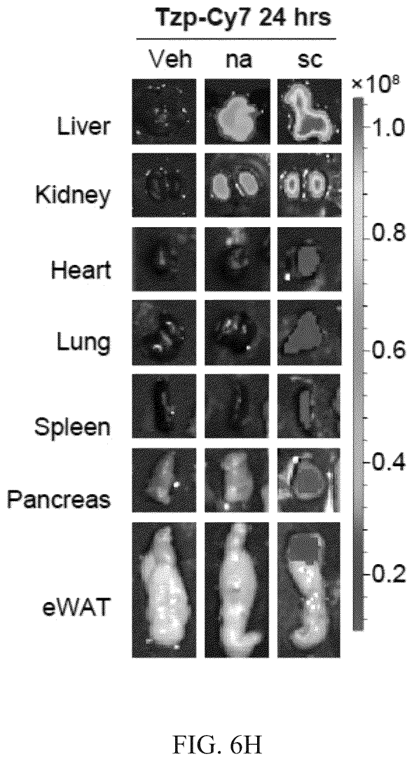

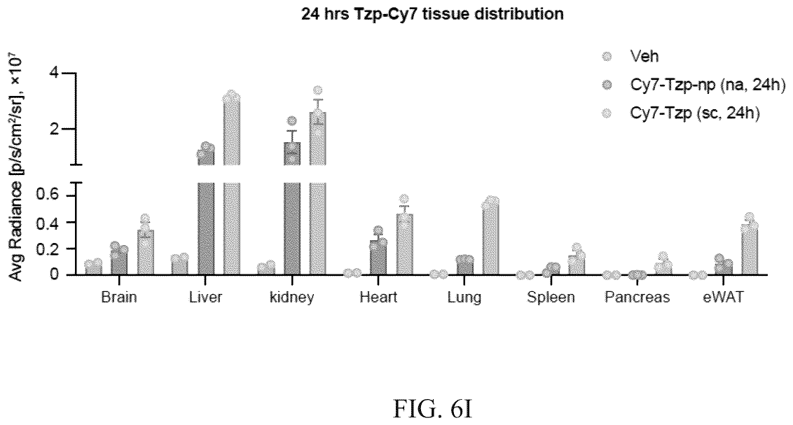

Intranasal delivery is known to primarily deliver to the olfactory bulb, which expresses abundant GLP-1 receptors. However, it is unknown whether GLP-1 could function in the olfactory bulb to affect appetite. Using fluorescent probe-labelled Tzp, Applicants did detect dominant Tzp-Cy5 signal in the olfactory bulb region within 1 hour post intranasal administration, in striking contrast to the minimal brain signal via subcutaneous injection (FIG. 6A). Intriguingly, both delivery routes potently suppressed appetite using fasted mice at 1 hour (FIG. 6B and FIG. 6C). Brain section imaging confirmed strong fluorescence signal in the olfactory regions and cerebral cortex by intranasal delivery route, while signal was barely detected in the subcutaneously injected mouse brain (FIG. 6D). Among peripheral tissues, only liver was detected Tzp-Cy5 signal in both delivery routes at 1 hour post administration (FIG. 6E), pointing to a central mechanism of Tzp to reduce food intake.

At 24 hours post intranasal administration, the Tzp fluorescence signal remained high in the olfactory bulb region while diffusing into other regions of the brain, whereas subcutaneous injection resulted in strong distribution to the brain, primarily the hypothalamus (FIG. 6F). Next, Applicants compared pharmacokinetics of Tzp via different delivery routes. The subcutaneous route showed quick entering of Tzp in the circulation, peaked at 5 hours and remained high at 24 hours post-injection (FIG. 6G), supporting the strong signals in peripheral organs (FIG. 6H). Notably, the plasma concentrations of intranasal delivered Tzp-Cy7 persisted at much lower levels over the 24-hr course, underlying its marginal peripheral tissue distributions (FIG. 6H and FIG. 6I). The relative stronger signal in the liver and kidney of intranasal delivery corresponds with the rapid clearance characteristic of nanoparticles. Overall, intranasal administration is an efficient route for delivering GLP-1 drugs to the brain and conveys appetite-suppressing function.

### Example 4-the Anti-obesity Effect of Chronic Intranasal Administration of Tzp-NPs

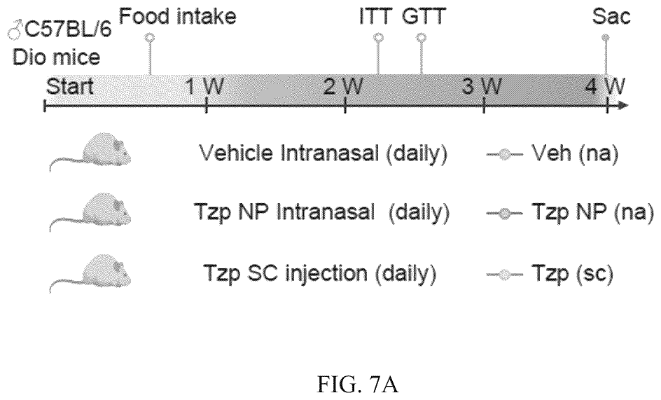

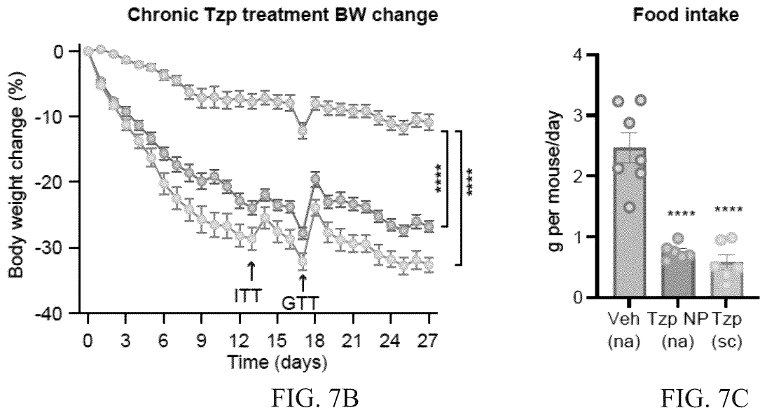

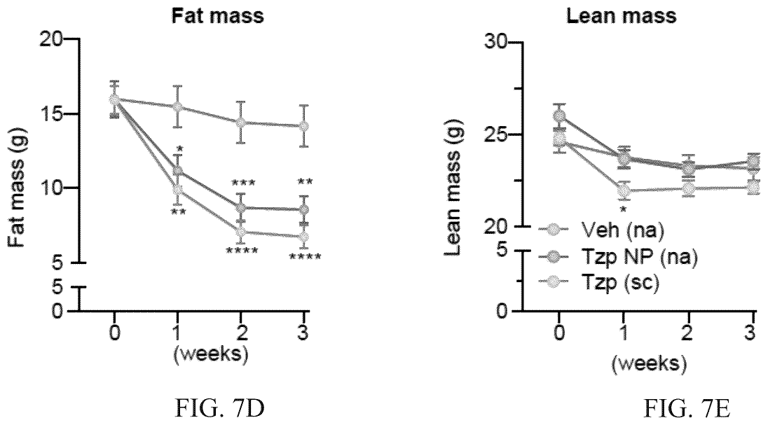

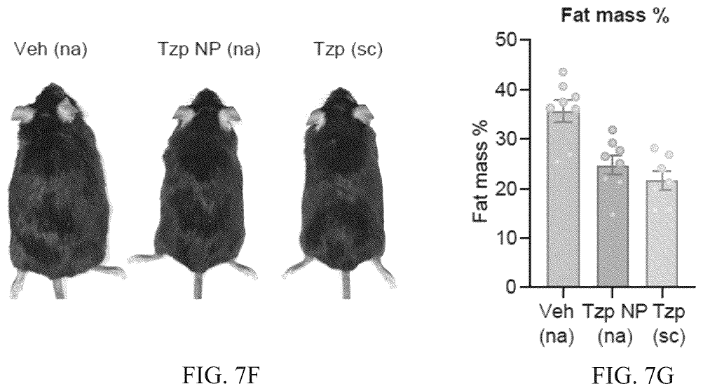

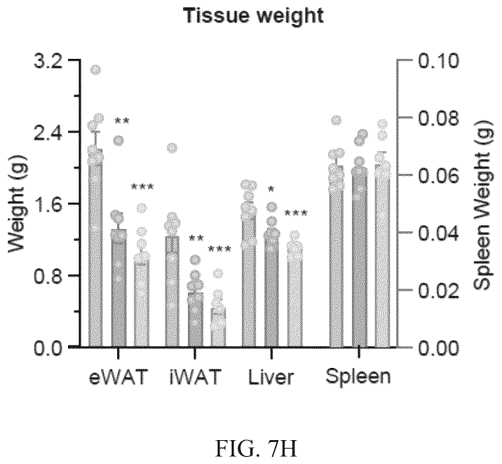

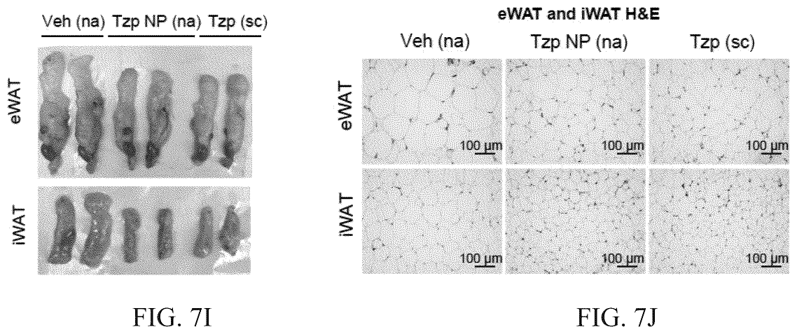

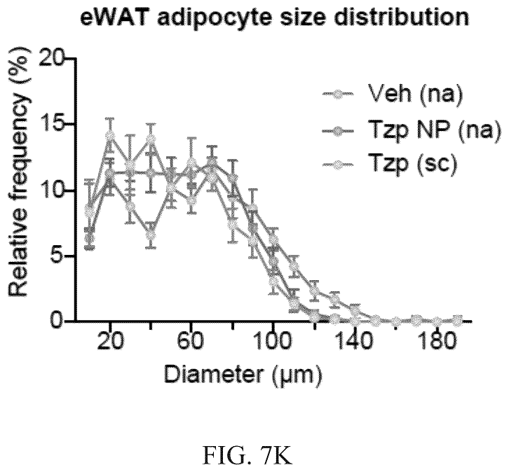

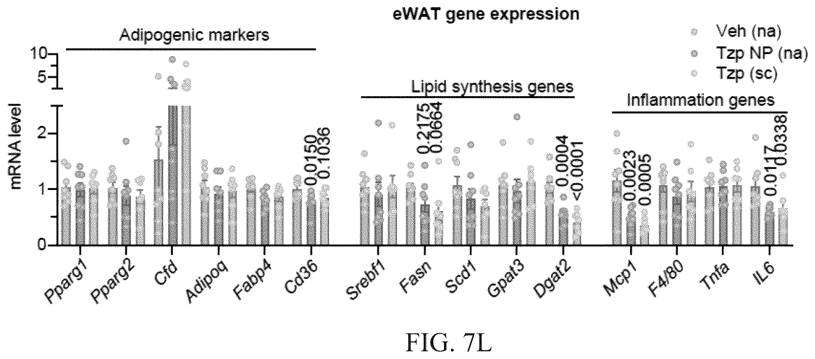

Applicants next proceeded to evaluate the long-term anti-obesity efficacy of intranasal Tzp-NP delivery in comparison to subcutaneous administration by treating DIO mice daily for 4 weeks (FIG. 7A). Both treatments quickly reduced body weight in the first two weeks and stayed mostly stable in the following two weeks, eventually losing 26.7% body weight for intranasal delivery and 32.6% for subcutaneous injection (FIG. 7B). The mild weight loss in the vehicle group is probably caused by the stress or single housing during the treatment. Recognizing that the major weight loss mechanism of GLP-1 drugs involves reducing food intake, Applicants proceeded to compare food consumption following the administration of Tzp via intranasal or subcutaneous routes. Food intake was decreased 70% and 80% in the intranasal and subcutaneous groups, respectively, compared to the vehicle group, and there was no significant difference between the two treatment groups (FIG. 7C), underpinning their weight loss. While both treatments potently decreased fat mass, subcutaneous injection was more potent in reducing both fat and lean mass (FIG. 7D and FIG. 7E). After four weeks, the resulted lean phenotype was supported by their significant decrease in fat composition (FIG. 7F and FIG. 7G). Intranasal treatment decreased visceral fat eWAT and subcutaneous iWAT undistinguishably by 50%, less than the 60% reduction by subcutaneous injection (FIG. 7H). The liver weight was also decreased in both Tzp-treated groups, without affecting the spleen size. Tzp treatment alleviated adipocyte hypertrophy in both eWAT and iWAT (FIG. 7I-FIG. 7L), usually an indicator of detrimental adipose function in obesity. Aligning with the lean phenotype, Dgat2, the gene encoding a key triglyceride synthesis enzyme, was downregulated in eWAT by Tzp treatment, with most adipocyte genes normally expressed (FIG. 7H). Obesity is typically associated with chronic inflammation in adipose tissue, and Tzp treatment downregulated the expression of inflammatory markers Mcp1 and Il6, regardless of the delivery routes, indicating alleviated adipose inflammation. In summary, these comparable results suggest that intranasal administration is an effective alternative for delivering Tzp in obesity treatment.

### Example 5-Intranasal Delivery of Tzp NPs Achieves Comparable Metabolic Improvements

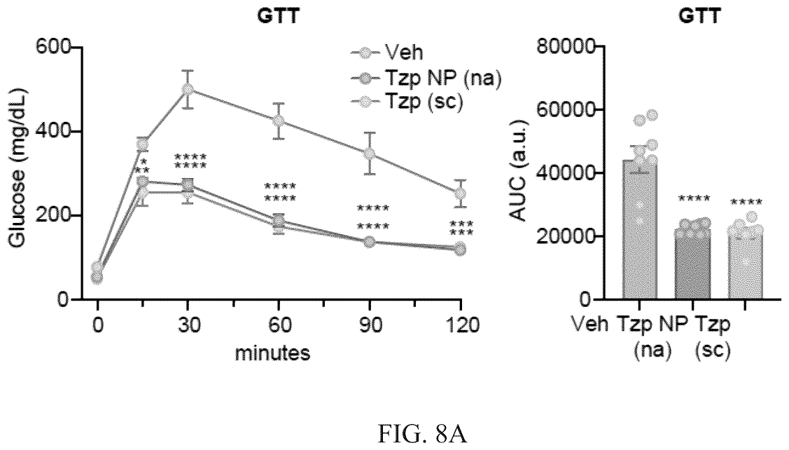

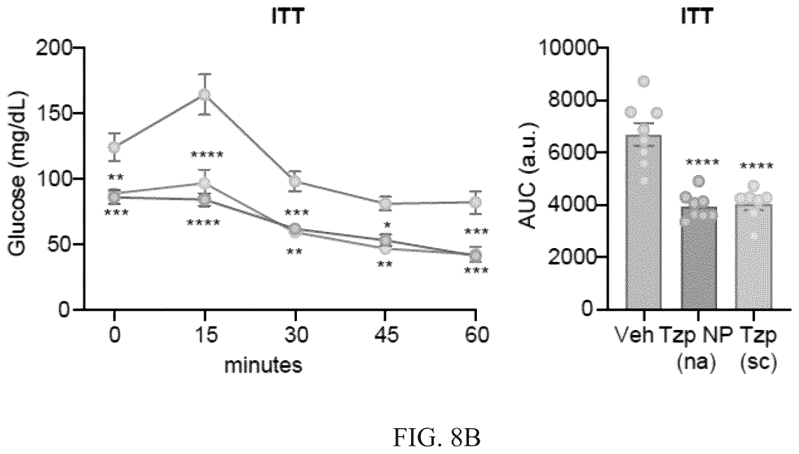

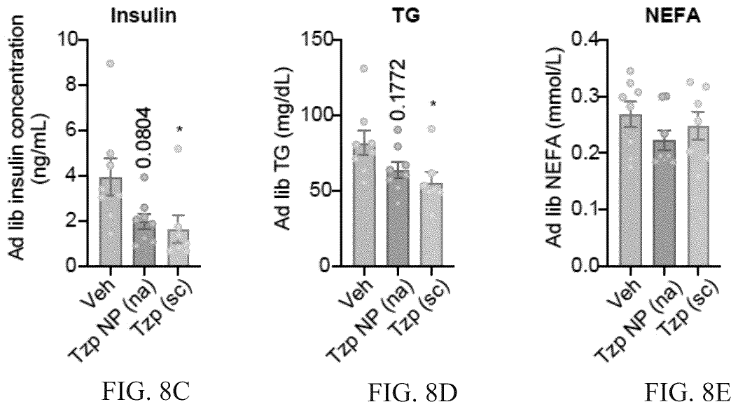

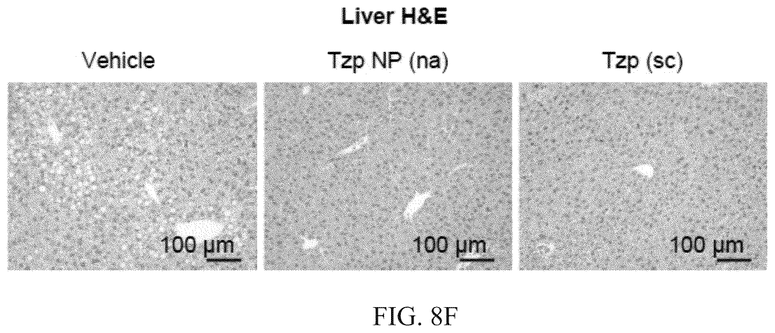

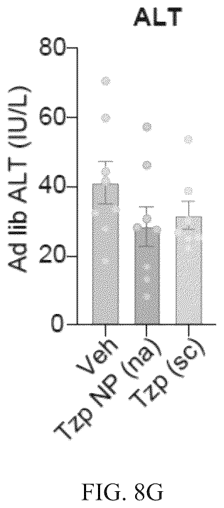

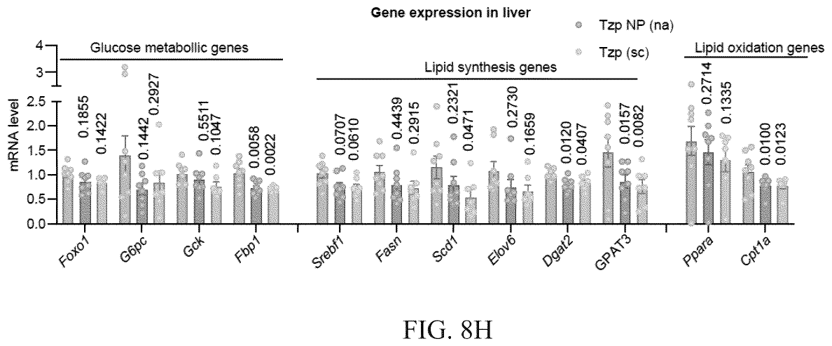

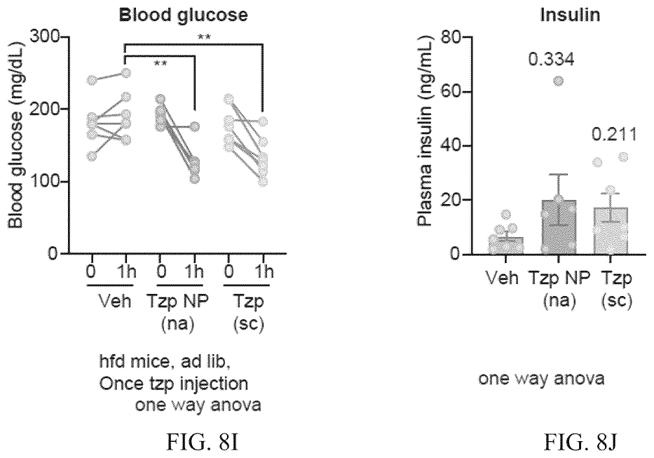

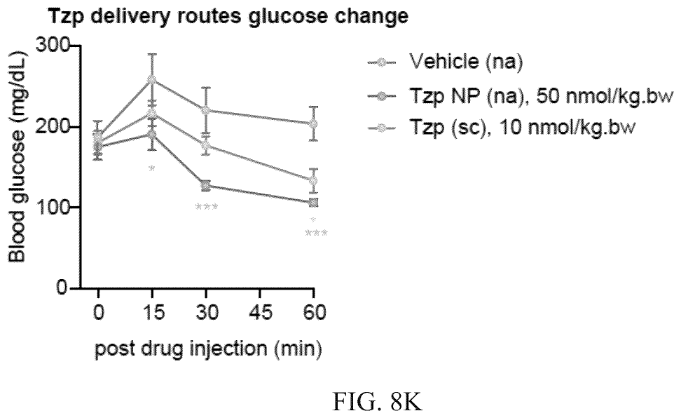

Beyond weight control, GLP-1 drugs also show attractive efficacy in treating diabetes and metabolic liver diseases, both of which are the primary comorbidities of obesity. Intranasal delivery of Tzp NPs showed the same strong improvements in glucose tolerance and insulin sensitivity as subcutaneous injection (FIG. 8A and FIG. 8B). In line with the enhanced insulin sensitivity, plasma insulin levels were decreased by chronic Tzp treatments (FIG. 8C). Intranasal treatment only mildly reduced plasma triglycerides (TG) and non-esterified fatty acids (NEFA) levels (FIG. 8D and FIG. 8E). Consistent with the reduced liver weight (FIG. 7H), both deliver routes improved liver steatosis (FIG. 8F), accompanied by trends of decreased plasma ALT levels, a marker of liver injury (FIG. 8G). The improved liver metabolic function is underlain by overall downregulations of genes associated with gluconeogenesis (Foxo1, G6pc, and Fbp1) and lipogenesis (Srebf1, Fasn, Scd1, Elov6, and Dgat2) (FIG. 8H).

GLP-1 RAs efficiently lower blood glucose levels through functioning in the pancreas to stimulate insulin release. Applicants then compared the acute effects of Tzp via different delivery routes. Intranasal delivery of Tzp NPs quickly decreased blood glucose levels within 1 hour, the same as subcutaneous injection (FIG. 8I), owing to the stimulation of insulin release (FIG. 8J). Interestingly, though it needs a higher dose to achieve comparable weight loss to subcutaneous injection administration (50 nmol/kg body weight verse 10 nmol/kg·bw), Tzp NPs were more efficient in lowering blood glucose levels (FIG. 8K), during which time Tzp primarily reached the olfactory bulb but not the pancreas (FIG. 6A and FIG. 6E), implicating a sympathetic neural pathway orientated in the olfactory bulb to stimulate insulin release.

### Example 6—Discussion

As a fundamental feature in living organisms, metabolic fluctuation is an essential way to achieve metabolic homeostasis. However, one high dose of long-acting GLP-1 drugs might flatten metabolic fluctuation despite its advantage in patient compliance. In contrast, intranasal delivery for short-term activation of GLP-1 might better mimic the fluctuations of GLP-1 production and function under physiological conditions.

Intranasal drug delivery has emerged as a promising route for treating various conditions, particularly those targeting the central nervous system. Specifically for peptide drugs, oral administration is often impractical due to rapid inactivation in the digestive system, leading to the common use of subcutaneous or intravenous administration. However, these routes present challenges such as inconvenience, pain, and infection risk, impacting patient adherence. In contrast, intranasal delivery offers a viable alternative. For instance, it enables rapid absorption, as demonstrated by glucagon nasal spray used for emergency hypoglycemia management. Moreover, intranasal delivery allows direct access to the brain, bypassing the bloodstream, making it a widely utilized approach in treating neurological disorders. Approved drugs like esketamine and desmopressin exemplify this, as they target the brain via intranasal administration. Additionally, intranasal delivery has been explored in trials involving GLP-1 drugs, including Exendin-4 and liraglutide, for the treatment of diabetes. However, the potential application of intranasal GLP-1 receptor agonists in managing obesity remains uncertain. In this study, Applicants found that intranasal administration of GLP-1 drugs, including but not limited to semaglutide, tirzepatide, and retatrutide, demonstrates similar effectiveness to subcutaneous delivery in treating obesity and associated metabolic disorders, particularly fatty liver conditions.

In this study, despite achieving comparable body weight loss and food intake suppression between intranasal administration and subcutaneous injection, particularly noticeable during the initial treatment days, there were differences in distribution across distinct brain regions. This inconsistency not only suggests distinct pharmacokinetics of TZP via different routes but also hints at the involvement of different neuronal circuits driven by GLP-1 in the brain. The olfactory bulb, known to be associated with appetite regulation (Stark R. The olfactory bulb: A neuroendocrine spotlight on feeding and metabolism. J Neuroendocrinol. 2024;36(6):e13382), also contains preproglucagon cells that locally release GLP-1 (Thiebaud N, Gribble F, Reimann F, Trapp S, Fadool D A. A unique olfactory bulb microcircuit driven by neurons expressing the precursor to glucagon-like peptide 1. Sci Rep. 2019; 9(1):15542; Montaner M, Denom J, Jiang W, Magnan C, Trapp S, Gurden H. The local GLP-1 system in the olfactory bulb is required for odor-evoked cephalic phase of insulin release in mice. Mol Metab. 2023;73:101738). Therefore, the high signal of TZP observed in the olfactory bulb following intranasal delivery may directly target the hypothalamus and brainstem through the circulation of cerebrospinal fluid (Born J, Lange T, Kern W, McGregor G P, Bickel U, Fehm H L. Sniffing neuropeptides: a transnasal approach to the human brain. Nat Neurosci. 2002;5(6):514-516). Whether there exists a cerebrospinal fluid-independent neuronal circuit from the olfactory bulb to the hypothalamus to regulate food intake remains unknown but is an intriguing possibility. The lower signal observed in the hypothalamus in the intranasal group not only suggests that the brain may not require high concentrations of GLP-1 for efficacy but also implies the existence of other neuronal circuits controlling food intake. Subcutaneous injection of tirzepatide resulted in sustained high plasma and organ levels even 24 hours post-injection. This dual nature of high plasma tirzepatide levels presents both advantages and disadvantages. While high concentrations contribute to rapid and effective body weight loss, prolonged exposure may lead to decreased sensitivity to GLP-1 drug over time, necessitating higher doses for long-term efficacy in humans. Additionally, the potential side effects of elevated tirzepatide concentrations in non-cerebral organs must be considered. Direct delivery to the brain via intranasal administration holds promise for circumventing systemic side effects. This study demonstrated better bone health and lower concentrations in the pancreas at 24 hours post-injection with intranasal delivery, compared to subcutaneous administration where acute pancreatitis has been reported in humans. However, it's important to note that mice may not fully replicate gastrointestinal side effects observed in human patients after GLP-1 drug administration; thus, Applicants did not observe any differences in stomach histology or gene expression of inflammatory markers (data not included). Further investigation is warranted to evaluate the benefits of nasal delivery in mitigating side effects, potentially utilizing primate models for a more accurate assessment.

Although the nasal route offers advantages as discussed above, drawbacks like nasal mucosal irritation and low bioavailability need to be considered. In this study, although there was some degree of body weight loss in the vehicle group following long-term PBS treatment, the nasal cavity exhibited normal morphology. This finding rules out nasal irritation as a side effect of long-term use. In this study, the bioavailability of intranasal GLP1 drugs is around 20% compared to subcutaneous administration, which aligns with previous reports (see, e.g., patent EP1696960B1). However, this low bioavailability may increase translation costs in clinical settings. Strategies to enhance nasal absorption could mitigate this issue. For instance, incorporating nanoparticles with absorption enhancers like cell-penetrating peptides and tight junction modulators could improve bioavailability via intranasal delivery. The presence of mucins in nasal mucus, which contain high levels of sialic acid and sulfate, imparts a strongly net-negative charge to the mucus surface. Applicants found that coating PLGA-TZP with the positively charged material chitosan significantly increased delivery efficiency. Moreover, further studies are warranted to explore methods for enhancing the efficiency of TZP entering the brain directly while minimizing systemic exposure to mitigate side effects. Additionally, prolonging the release of nanoparticles in the brain could extend therapeutic efficacy, reduce treatment frequency, and improve patients'quality of life. These endeavors represent meaningful avenues for future research in intranasal drug delivery optimization in obesity management.

Taken together, the current study offers an alternative, painless, easy self-administration, and non-invasive method for delivering GLP-1 drugs to address obesity and its associated metabolic disorders.

### Example 7—Methods

GLP-1 drug nanomedicine preparation. Tzp was encapsulated into PLGA nanoparticles which were prepared via double emulsion as previous literature has described (Saffarionpour S, One-step preparation of double emulsions stabilized with amphiphilic and stimuli-responsive block copolymers and nanoparticles for nutraceuticals and drug delivery. JCIS Open. 2021; Volume 3,100020). TZP stock solution was prepared by dissolving Tzp (Peptide Sciences, C A) in ddH₂O and kept on ice or at 4° C. PLGA polymer (Resomer® RG 504 H, Sigma-Aldrich®, M A) was dissolved in chloroform and was prepared fresh in every batch. Surfactant sodium dodecyl sulfate (SDS, Fisher BioReagents®, P A) was used to stabilize droplets during emulsion. SDS solution was prepared in dd water. The double emulsion process took place in two steps. First, the Tzp stock solution (aqueous, 10 mg/ml) and the PLGA solution (organic, 10 mg/ml) were mixed at the volume ratio of 1:4 and sonicated with a 20 kHz ultrasonic processor to create aqueous droplets in the organic phase, which appeared untransparent to the naked eyes. This step is also called a water-in-oil step. Immediately after the first step, the SDS solution was added to the previous mixture at 4× the volume of the PLGA solution. The new mixture was then sonicated with the ultrasonic processor, which is also known as a water-in-oil-in-water emulsion. The organic solvent was removed by rotary evaporation. Afterward, the nanoparticles were concentrated by using Amicon ultracentrifugation filter units (MilliporeSigma™, Darmstadt, Germany) with Mwco 10 kDa. SDS was removed by washing with dd water three times. The resulting nanoparticles were lyophilized and kept at −20° C. for later usage.

In order to coat the Tzp-loaded NPs with additional polymers, a technique called flash nanocomplexation (FNC) was used (Hu H) Yang C, Li M, Shao D, Mao H Q, Leong K W. Flash Technology-Based Self-Assembly in Nanoformulation: From Fabrication to Biomedical Applications. Mater Today (Kidlington). 2021;42:99-116). Briefly put, because the Tzp NPs have negative surface charges, cationic polymers can be coated onto these nanoparticles via electrostatic interactions. With FNC, the nanoparticles and the polymer solution (aqueous) were infused through two different channels but turbulently mixed together within a confined chamber. As a result, a homogeneous coating layer was generated. The ratio and concentrations were variable and depended on the goal regarding the final surface charge.

Characterization of nanoparticles. The morphologies of the NPs were characterized using a FEI Talos transmission electron microscope (Thermo Fisher™, M A). The hydrodynamic diameter and zeta potential of the NPs in water or PBS were characterized using a Nano-ZS 90 Nanosizer (Malvern Instruments Ltd., Worcestershire, U K).

For the drug release test, the lyophilized TZP-loaded nanoparticles were dispersed in PBS and gently shaken at 100 rpm at 37° C. At predetermined time points (0, 0.5, 1, 2, 3, 4, 5, 6, 8, 10, 24, 48, 72, 96 hr), the amount of released peptide in the supernatant was measured. The amount of peptide was quantified by Pierce BCA protein assay kits (Thermo Scientific®, M A).

Mice administration. The mice used in the study were bred from the C57BL/6J strain and were housed in the animal facility at Columbia University. They were kept in a controlled environment with a temperature maintained at 23±1° C. and subjected to a 12-hour light-dark cycle. The mice had free access to standard chow food (PicoLab® Rodent 5053) and water. For experiments involving a high-fat diet (HFD), a diet consisting of 60% fat was obtained from Research Diets® (D12492i). 25-week-old male mice were given a high-fat diet (HFD) for six weeks to induce obesity. For short-term studies, diet-induced obese (DIO) mice were administered daily treatments of semaglutide, tirzepatide, and retatrutide via subcutaneous injection, or their PLGA nanoparticle formulations via intranasal injection, for 6 days, 3 days, and 7 days, respectively. A control group was administered water intranasally. Food intakes were measured for the first three days following tirzepatide treatment. For long-term tirzepatide study, following a two-week period where body weight reached stability, metabolic analyses like ITT and GTT measurements commenced. Body weight was tracked daily, and body composition was assessed weekly using EchoMRI. Four weeks after the initial injection, the mice were euthanized at ad libitum conditions. For drug administration, mice were anesthetized with isoflurane and maintained in a surgical plane of anesthesia. Following anesthesia induction, mice received either a subcutaneous or intranasal injection of the test drug. For intranasal administration, a volume of 5 μL of the drug solution in water was delivered to each mouse. Dosages were 10 nmol/kg for subcutaneous treatments and 50 nmol/kg for intranasal treatments.

Metabolic profiling. For the glucose tolerance test (GTT), mice were fasted for 16 hours in clean cages with fresh bedding before receiving a 2 g/kg body weight intraperitoneal (i.p.) injection of glucose. Blood glucose levels were measured using a Breeze® 2 glucometer (Bayer®) at 0, 15, 30, 60, 90 and 120 minutes post-injection via tail vein bleeding. For the insulin tolerance test (ITT), mice fasted for 4 hours were then administered an i.p. injection of insulin at a dosage of 0.75 U/kg body weight. Blood glucose levels were measured at 0, 15, 30, 45, and 60 minutes post-injection. For measuring food intake, mice were individually housed. Their high-fat diet was placed in a stainless container to which they had unrestricted access, along with water. The food container was weighed daily to track consumption.

Serum NEFA (Fujifilm® Wako), T G (Thermo Scientific®), insulin (Mercodia® Insulin ELISA), and alanine aminotransferase (ALT, Teco Diagnostics A526120) levels were quantified in accordance with the respective manufacturer's instructions.

Dye-labelled tirzepatide's in vivo tissue distribution. Tirzepatide was conjugated with Cy5 or Cy7 and encapsulated into PLGA nanoparticles. Mice received these labeled compounds via subcutaneous or intranasal injections at doses of 10 nmol/kg.bw or 50 nmol/kg.bw, respectively. In vivo imaging was conducted at 1, 2, 3, and 24 hours post-injection with the PerkinElmer® IVIS® system. Following euthanasia, tissue signals were quantified at 3 and 24 hours post-injection using the same system. Blood was collected from the tail vein at 1, 2, 3, 5, 8, and 24 hours post-injection, and the Cy5 fluorescence signal in plasma was analyzed using a SpectraMax® M2 plate reader (Molecular Devices®, C A).

Bone processing and analysis. Femurs and tibias were harvested and fixed in 10% neutral buffered formalin at 4° C. overnight, then analyzed for bone micro-architecture and lipid content. The micro-architecture was scanned using a Quantum FX μCT Scanner. Bone mineral density (BMD) were measured at consistent 60 slice intervals from the femur's or tibia's growth plate. For lipid analysis, bones were decalcified in 14% EDTA, at 4° C., for at least 2 weeks, with the solution changed every 3 to 4 days. Then, bones were stained with 1% osmium tetroxide and 2.5% potassium dichromate for 48 hours and further imaged by μCT. Marrow adipose sections were identified at consistent 250 slice intervals from the femur's or tibia's growth plate. Analyze 12 software quantified lipid volumes, while Analyze 14 determined bone mineral density.

Gene expression. RNA from tissues was extracted using the Tri-Isolate RNA pure kit (IBI Scientific™) following the manufacturer's guidelines. Subsequently, 1,000 ng RNA underwent reverse transcription using the high-capacity cDNA reverse transcription kit (Applied Biosystems®) to produce cDNA. Quantitative real-time PCR (qPCR) was then conducted using the Bio-Rad® CFX96 real-time PCR system and GoTaq® qPCR Master Mix (Promega®). The relative gene expression was assessed using the ΔΔCt method, with Rpl23 or Cpa serving as reference genes. Primer sequences were available upon request.

Histology. Following dissection, epididymal white adipose tissue (eWAT), inguinal white adipose tissue (iWAT), and liver were collected and immediately fixed by immersion in 10% formalin solution. Subsequently, tissues were then embedded in paraffin, sectioned, and stained with hematoxylin and eosin (H&E) to prepare for subsequent microscopic examination. For brain immunohistology, mice were first perfused with PBS, then switched to 10% buffered formalin to fix the brain. A cFOS antibody was used. Immunohistochemical staining was performed on stomach sections using primary antibodies against Atp4b (Abcam®) and Ki67 (Cell Signaling Technology®) following established protocols.

For histological analysis of the nasal cavity, the following procedure was used. Following euthanasia, the heads of mice were collected and carefully skinned. The lower jaw was then discarded. The remaining head tissues were immersed in 10% buffered formalin solution for fixation for 48 hours. Following fixation, decalcification was performed using a 10% EDTA solution for 20 days, with the solution refreshment every 3-4 days. Decalcified tissues were then processed for paraffin embedding, sectioning, and H&E staining for histological evaluation of the nasal cavity.

Statistical analysis. The significance of the differences between groups of mice was assessed using two-way ANOVA. A paired t-test was employed to evaluate the significance of body weight changes before and after treatment. A p-value of less than 0.05 was considered statistically significant. Data in this study are presented as mean±standard error of the mean (s.e.m.). Analysis was conducted using Prism 9.3.1 (GraphPad®).

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

We claim:

1. A method of delivering a therapeutic agent having GLP-1 receptor agonist activity to a patient, the method comprising intranasal delivery of a pharmaceutical composition comprising the therapeutic agent having GLP-1 receptor agonist activity and a nanoparticle delivery system comprising polymeric nanoparticles, wherein the nanoparticle delivery system encapsulates the therapeutic agent having GLP-1 receptor agonist activity.

2. The method of claim 1, wherein the therapeutic agent having GLP-1 receptor agonist activity is selected from the group consisting of tirzepatide, semaglutide, and retatrutide.

3. The method of claim 1, wherein the therapeutic agent is present in an amount effective to treat obesity, an obesity-related comorbidity, hyperglycemia, fatty liver disease, or a related metabolic disorder.

4. The method of claim 1, wherein the nanoparticle delivery system comprises polymeric nanoparticles comprising poly(lactic-co-glycolic acid) (PLGA).

5. The method of claim 1, wherein the nanoparticle delivery system comprises polymeric nanoparticles comprising poly(caprolactone) (PCL) nanoparticles.

6. The method of claim 5, wherein the nanoparticle delivery system further comprises a coating comprising at least one positively charged biocompatible polymer, wherein the polymeric nanoparticles are coated with the coating.

7. The method of claim 6, wherein the positively charged biocompatible polymer is selected from chitosan (CS) and polyethylenimine-grafted chitosan (CS-PEI).

8. The method of claim 7, wherein the nanoparticle delivery system comprises polymeric nanoparticles comprising PLGA nanoparticles coated with CS-PEI.

9. The method of claim 5, wherein the nanoparticle delivery system comprises polymeric nanoparticles comprising poly(caprolactone) (PCL) nanoparticles coated with chitosan (CS) or polyethylenimine-grafted chitosan (CS-PEI).

10. The method of claim 1, wherein the GLP-1 drug is delivered to the patient's olfactory bulb.

11. The method of claim 6, wherein the coating-to-PLGA ratio is 0.25.

12. The method of claim 6, wherein the coated polymeric nanoparticle has a polydispersity index (PdI) of 0.10±0.02 or 0.15±0.05.

13. The method of claim 4, wherein the polymeric nanoparticle has a polydispersity index (PdI) of 0.15±0.05.

14. The method of claim 6, wherein the coated nanoparticle has a zeta potential of 49.3±1.3 mV.

15. The method of claim 1, wherein the polymeric nanoparticle has a hydrodynamic diameter of 110 nm-130 nm.

16. A method of treating obesity, an obesity comorbidity, or any other condition causing or associated with high blood glucose levels or fatty liver in a subject comprising intranasally administering a pharmaceutical composition comprising a therapeutic agent having GLP-1 receptor agonist activity and a nanoparticle delivery system comprising polymeric nanoparticles, wherein the nanoparticle delivery system encapsulates the therapeutic agent having GLP-1 receptor agonist activity.

17. The method of claim 16, wherein the nanoparticle delivery system comprises poly(lactic-co-glycolic acid) (PLGA) nanoparticles.

18. The method of claim 16, wherein the nanoparticle delivery system comprises poly(lactic-co-glycolic acid) (PLGA) nanoparticles coated with at least one positively charged biocompatible polymer selected from chitosan (CS) and polyethylenimine-grafted chitosan (CS-PEI).

19. A pharmaceutical composition comprising:
a GLP-1 drug; and
a nanoparticle delivery system, wherein the nanoparticle delivery system comprises poly(lactic-co-glycolic acid) (PLGA) or poly(caprolactone) (PCL) nanoparticles.

20. The pharmaceutical composition of claim 19, further comprising a coating comprising at least one positively charged biocompatible polymer selected from chitosan (CS) and polyethylenimine-grafted chitosan (CS-PEI), wherein the PLGA or PCL are coated with the coating.
